(19) United States
(12) Patent Application Publication
(10) Pub. No.: US 2025/0152939 A1
(43) Pub. Date: May 15, 2025

(54) SYSTEMS AND METHODS FOR PROMOTING GENERAL WELLNESS

(71) Applicant: ElectroCore, Inc., Rockaway, NJ (US)

(72) Inventors: Bruce J. Simon, Wellfleet, MA (US); Joseph P. Errico, Lake Worth, FL (US); Peter Staats, Atlantic Beach, FL (US); Jonathan Gardiner, Budd Lake, NJ (US); Jeckin Shah, Flemington, NJ (US); Michael Romaniw, Mount Pocono, PA (US)

(21) Appl. No.: 18/935,523

(22) Filed: Nov. 2, 2024

Related U.S. Application Data
(60) Provisional application No. 63/598,994, filed on Nov. 15, 2023.

(51) Int. Cl. A61N 1/36; A61N 1/04

(52) U.S. Cl. CPC A61N 1/36034; A61N 1/0456; A61N 1/36025

(57) ABSTRACT

Systems, devices and methods are provided for stimulating one or more nerves within a user. A system comprises stimulator having an electrode configured for contacting the outer skin surface at a target location and an energy source coupled to the stimulator, The energy source is configured to generate at least one electrical impulse and to transmit the electrical impulse transcutaneously from the electrode through the outer skin surface of the user to a selected nerve in the user. In certain aspects, the systems and methods are particularly useful for non-medical purposes, such as reducing stress and anxiety, enhancing relaxation, improving sleep, energy, concentration and mood, increasing mental or physical performance, promoting mental health, recovery and wellness, generally improving the health and wellbeing of a user and/or for temporarily or permanently improving intelligence, learning capacity, memory retention, recall, mood, alertness and/or sleep efficiency in human beings.

### BACKGROUND

The use of electrical stimulation for treatment of medical conditions is well known. One of the most successful applications of modern understanding of the electrophysiological relationship between muscle and nerves is the cardiac pacemaker. Although origins of the cardiac pacemaker extend back into the 1800's, it was not until 1950 that the first practical, albeit external and bulky, pacemaker was developed. The first truly functional, wearable pacemaker appeared in 1957, and in 1960, the first fully implantable pacemaker was developed.

Many therapeutic applications of electrical stimulation involve the surgical implantation of electrodes within a user. Recently, minimally invasive electrical stimulators that transmit energy to nerves non-invasively or percutaneously have become more common. A medical procedure is defined as being non-invasive when no break in the skin (or other surface of the body, such as a wound bed) is created through use of the method, and when there is no contact with an internal body cavity beyond a body orifice (e.g., beyond the mouth or beyond the external auditory meatus of the ear). Such non-invasive procedures are distinguished from invasive procedures (including minimally invasive procedures) in that the invasive procedures insert a substance or device into or through the skin (or other surface of the body, such as a wound bed) or into an internal body cavity beyond a body orifice.

Non-invasive medical methods and devices provide a number of advantages relative to comparable invasive procedures. For example, the user or user may be more psychologically prepared to experience a procedure that is non-invasive and may therefore be more cooperative, resulting in a better outcome. Non-invasive procedures may avoid damage of biological tissues, such as that due to bleeding, infection, skin or internal organ injury, blood vessel injury, and vein or lung blood clotting. Non-invasive procedures can be generally measurably painless and may be performed without some of the dangers and costs of surgery. They are ordinarily performed even without the need for local anesthesia. Less training may be required for use of non-invasive procedures by medical professionals. In view of the reduced risk ordinarily associated with non-invasive procedures, some such procedures may be suitable for use by the user or family members at home or by first-responders at home or at a workplace. Furthermore, the cost of non-invasive procedures may be significantly reduced relative to comparable invasive procedures.

In some cases, the user or user can apply the stimulator without the benefit of having a trained healthcare provider nearby. An advantage of this “self-stimulation” therapy is that it can be administered more or less immediately when symptoms occur, rather than having to visit the healthcare provider at a clinic or emergency room. A need for such a visit would only compound the aggravation that the user is already experiencing. Another advantage of the self-stimulation therapy is the convenience of providing the therapy in the user's home or workplace, which eliminates scheduling difficulties, for example, when the nerve stimulation is being administered for prophylactic reasons at odd hours of the day. Furthermore, the cost of the treatment may be reduced by not requiring the involvement of a trained healthcare provider.

### SUMMARY

Systems, devices and methods are provided for delivering energy impulses (and/or fields) to bodily tissues for various purposes. In certain aspects, the systems and methods are particularly useful for non-medical purposes, such as reducing stress and anxiety, enhancing relaxation, improving sleep, energy, concentration and mood, increasing mental or physical performance, promoting mental health, recovery and wellness and generally improving the health and wellbeing of a user. The system and methods may also be useful for improving a cognitive performance of the user by temporarily or permanently improving intelligence, learning capacity, memory retention, recall, mood, alertness and/or sleep efficiency.

In one aspect, a system for stimulating a nerve within a user comprises a stimulator comprising an electrode configured for contacting the outer skin surface at, or near, the target location and an energy source coupled to the stimulator. The energy source is configured to generate at least one electrical impulse and to transmit the at least one electrical impulse transcutaneously from the electrode through the outer skin surface of the user to a selected nerve in the user adjacent to, or near, the target location. The electrical impulse is sufficient to improve a general wellness of the user. Improving the general wellness may include, but is not limited to, reducing stress and anxiety, enhancing relaxation, improving sleep, energy, concentration and mood, increasing mental or physical performance, promoting mental health, recovery and wellness and generally improving the overall health and wellbeing of a user.

In various embodiments, the electrical impulse is sufficient to reduce a stress of the user. In other embodiments, the electrical impulse is sufficient to reduce anxiety of the user. In other embodiments, the electrical impulse is sufficient to increase an energy level of the user.

In various embodiments, the device further comprises a housing, such as a handheld device, that may be operated by the user. The energy source is housed within the housing and the electrodes are attached to, or incorporated into, the housing.

The housing may contain the electronic components, signal generator and energy source (not shown) that are used to generate the signals that drive electrical impulses through the electrodes. However, in other embodiments, the electronic components that generate the signals may be in a separate housing or device, such as a mobile device. Furthermore, other embodiments may contain a single electrode or more than two electrodes.

In an exemplary embodiment, the housing comprises a power button disposed on a surface of the housing, wherein the power button comprises a user interface. The power button comprises a substantially circular main body with first and second arms extending therefrom. The first and second arms comprise openings therethrough.

In various embodiments, the housing further comprises a support member between the power button and the housing. The support member comprises a main body having at least one support arm extending therefrom. The support member comprises a first surface coupled to the power button. The first surface tapers inwardly toward the housing. The support member further comprises a second surface coupled to the housing. The second surface comprises a recessed portion.

In another aspect, a system for stimulating a nerve within a user comprises a stimulator comprising an electrode configured for contacting the outer skin surface at, or near, the target location and an energy source coupled to the stimulator. The energy source is configured to generate at least one electrical impulse and to transmit the at least one electrical impulse transcutaneously from the electrode through the outer skin surface of the user to a selected nerve in the user adjacent to, or near, the target location. The electrical impulse is sufficient to improve a cognitive performance of the user. Improving cognitive performance may include, but is not limited to, temporarily or permanently improving intelligence, learning capacity, memory retention, recall, mood, alertness and/or sleep efficiency in human beings.

In various embodiments, the electrical impulse is sufficient to increase a memory of the user.

In various embodiments, the electrical impulses is sufficient to reduce a fatigue of the user.

In various embodiments, the electrical impulse is sufficient to increase a language acquisition skill of the user.

In various embodiments, the electrical impulse is sufficient to increase an attention span of the user.

In various embodiments, the electrical impulse is sufficient to increase a focus of the user.

In various embodiments, the system further comprises a software application configured for downloading onto a user interface. The software application controls parameters of the stimulator, which may be based on a physiological parameter of the user and/or user status information related to the effectiveness of the therapy.

In embodiments, the system further comprises one or more sensors configured to sense a physiological parameter of the user. The physiological parameter may include blood flow associated with a nerve, heart rate or variability, ECG, respiration status, depth and rate, core temperature, hydration, blood pressure, brain function, oxygenation, skin impedance, and skin temperature, pupil diameter or dilation, galvanic skin response, selected biomarkers, a property of a voice of the user, a laryngeal electromyographic signal, an electroglottographic signal and a property of the autonomic nervous system.

The software application may control the user interface to prompt a user to enter user status information and to compare the user status information with the physiological parameter. The user status information may include any data or information related to the effectiveness of the therapy, such as the level of pain experienced by the user, the user's satisfaction level, his or her mood, recent medication use, particularly pain medication, the user's perceived activity level, the amount of sleep that the user has received or other data related to the overall effectiveness of the therapy.

In certain embodiments, the software application controls the user interface to prompt a user to enter user status information and to compare the user status information to the parameters of the stimulator. The parameters may include an amplitude, frequency, duration of waveform of the electrical impulse.

In certain embodiments, the software program is configured to determine the usage levels based on a period of time that the one or more electrical impulses are generated by the signal generator and applied to the one or more electrodes. This allows the user and/or the caregiver to track usage of the stimulator and compare this usage with, for example, a prescribed therapy regimen.

The system may further include a controller or processor coupled to the user interface, and a second software application that compiles the user status data that has been input by the user into an aggregate set of data that provides valuable information on the status of the user. The second processor may be disposed on the stimulation device, the mobile device or an external processing device coupled to the mobile device. Since this information can be inputted by the user throughout the therapy regimen, it provides historical data for the user to understand how his/her status has changed throughout the therapy, i.e., pain levels have consistently gone down, medication use has decreased, etc.

In certain embodiments, the software application compares the user status data to the certain parameters of the device, such as usage levels, parameters of the stimulation protocol and the like. Comparing usage levels of the device directly with the user status data and/or parameters of the stimulation protocol allows the user to directly correlate usage of the device with his/her status at the time of such usage, e.g., a reduction in pain or a decreased use of medication to alleviate pain may directly correlate with usage. Providing this direct correlation provides confidence to the user that the therapy regimen is effective, and may improve user compliance.

In one such embodiment, the stimulator is configured to apply a plurality of single doses, wherein each single dose comprises applying the electrical impulse for a duration of between about 30 seconds and 5 minutes. The parameters controlled by the software application include a number of single doses applied by the stimulator, either in total, or over a time period, such as doses/day, doses/week, doses/month and the like.

In embodiments, the software application is configured to adjust the parameters of the stimulator based on the user status information. The software application may adjust these parameters automatically or it may provide an alert based on the user status information prompting the user to adjust the parameters of the stimulator.

In certain embodiments, the electrical impulse comprises pulses having a frequency of about 1 kHz to about 20 KHz. The electrical impulse may comprise bursts of pulses, with each burst having a frequency of about 1 to about 100 bursts per second and each pulse has a duration of about 50 to about 1000 microseconds in duration. The bursts each comprise about 2 to 20 pulses and the bursts are separated by an inter-burst period that comprises zero pulses.

In other embodiments, the device comprises a patch having at least one adhesive surface for attachment to the outer skin surface of the neck of the user. The electrodes are housed within the patch. The patch may further comprise a signal generator and an energy source for applying the electrical impulses through the electrodes to the vagus nerve. Alternatively, the patch may include a wireless receiver and associated electronics for wirelessly receiving the electrical impulse and/or the energy from the energy source.

The device may further comprise a controller coupled to the energy source and configured to transmit parameters for the stimulation protocol to the energy source. The controller and/or the energy source may be wirelessly coupled to the electrodes, or each other. Alternatively, the controller and the energy source may be housed within the patch or the handheld device.

In one embodiment, the electrical impulse is transmitted for at least 30 seconds within 4 hours of a commencement of symptoms in the user. The electrical impulse may be applied in a single dose for a time period of about 30 seconds and about 5 minutes, preferably about 90-150 seconds, or it may be applied in a series of doses each having a time period of about 30 seconds to about 3 minutes, preferably about 90-150 seconds in each dose. The series of doses may be applied every 5 to 30 minutes, preferably every 10 to 20 minutes, and more preferably every 15 minutes, for a period of at least 1 hour, preferably at least 2 hours and more preferably about 3 hours. Each dose may be further applied every 6 to 10 hours for a period of at least 2 to 10 days, preferably about 2 to 5 days.

In another embodiment, the electrical impulse is applied in a first dose for a time period of about 30 seconds and about 3 minutes, preferably about 90-150 seconds and then a second dose for a time period of about 30 seconds and about 3 minutes, preferably about 90-150 seconds. The electrical impulse may be transmitted in a series of first and second doses, wherein the electrical impulse is applied for a time period of about 30 seconds to about 3 minutes (preferably about 90-150 seconds) in each of the first and second doses. The first and second doses may be applied every 10 to 30 minutes (preferably about every 15 minutes) for a period of at least at least 1 hour, preferably at least 2 hours and more preferably about 3 hours. Each dose may be further applied every 6 to 10 hours for a period of at least 2 to 10 days, preferably for about 2 to 5 days.

In certain embodiments, the energy source is wirelessly coupled to the one or more electrodes. In other embodiments, the energy source is coupled to the electrodes directly with electrical connectors. In yet other embodiments, the energy source and the electrodes are housing within a handheld device that can be placed or attached against the outer surface of the user's neck.

In one such embodiment, the electrodes are adhered to the outer skin surface of the user's neck with a suitable adhesive. This allows the user to be treated without direct intervention (i.e., holding a device or the electrodes against the user's neck during stimulation). The system may further comprise an outer sheath or other wearable device, such as an insulating strip, a collar, or a garment, such as a turtleneck, a scarf, neck massager, neck pillow or the like, that functions to adhere or otherwise position the electrodes to the neck of the user. The electrodes may be housed within the wearable device, or positioned between the wearable device and the neck of the user.

In certain embodiments, the housing comprises upper and lower portions and a cover disposed between the upper and lower portions for protecting the electrodes from the external environment. The cover also ensures that electrodes will not contact a user's tissue when the device is not intended to be used (e.g., in the event that the device is accidently turned ON and electric current is passed through electrodes when not in use). In certain embodiments, the cover is rotatably coupled to the housing such that it can be moved between a first position, wherein the electrodes are exposed for stimulation, and a second position, wherein the electrodes are housed and protected within the cover.

The housing may comprise a user control panel that includes a number of user controls and/or device status indicators. In alternative embodiments, the controls and status indicators are located on a separate device, such as a mobile device, that is wirelessly (e.g. Bluetooth or the like) coupled to housing.

Various technologies for stimulating nerves are more completely described in the following detailed description, with reference to the drawings provided herewith, and in claims appended hereto. Other aspects, features, advantages, etc. will become apparent to one skilled in the art when the description is taken in conjunction with the accompanying drawings.

### INCORPORATION BY REFERENCE

Hereby, all issued patents, published patent applications, and non-patent publications that are mentioned in this specification are herein incorporated by reference in their entirety for all purposes as if copied and pasted herein, to the same extent as if each individual issued patent, published patent application, or non-patent publication were specifically and individually indicated to be incorporated by reference and copied and pasted herein.

### DESCRIPTION OF DRAWINGS

FIG. 1 illustrates a schematic view of one embodiment of a nerve modulating system;

FIG. 2A shows an embodiment of an electrical voltage/current profile for stimulating and/or modulating impulses that are applied to a nerve;

FIG. 2B illustrates an embodiment of a bursting electrical waveform for stimulating and/or modulating a nerve;

FIG. 2C illustrates an embodiment of two successive bursts of the waveform of FIG. 2B;

FIG. 3A is a perspective view of a stimulator;

FIG. 3B is a perspective view of the stimulator of FIG. 3A flipped upside down;

FIG. 3C is a perspective view of another embodiment of a stimulator with a cover for protecting the electrodes;

FIG. 3D is a perspective view of the stimulator of FIG. 3C with the cover positioned to expose the electrodes;

FIG. 4 is a perspective view of another embodiment of a stimulator;

FIG. 5A is a perspective view of another embodiment of a stimulator in the closed position;

FIG. 5B is a perspective view of the stimulator of FIG. 5A in an open position;

FIG. 5C is a perspective view of another side of the stimulator of FIG. 5A in the closed position;

FIG. 5D is a perspective view of another side of the stimulator of FIG. 5A in the open position;

FIG. 6A is a front view of another embodiment of a stimulator;

FIG. 6B is a back view of the stimulator of FIG. 6A;

FIG. 6C is a side view of the stimulator of FIG. 6A;

FIG. 6D is a bottom view of the stimulator of FIG. 6A;

FIG. 7A is a perspective view of a power button of the stimulator of FIG. 6A;

FIG. 7B is a front view of the power button;

FIG. 8A is a front view of a support member for the power button;

FIG. 8B is a back view of the support member;

FIG. 9 shows an expanded diagram of an embodiment of a control unit;

FIG. 10 illustrates an embodiment of an approximate position of a stimulator when used to stimulate a right vagus nerve in a neck of a user;

FIG. 11 illustrates a system for modulating the vagus nerve;

FIG. 12 illustrates a patch stimulator device for attaching to a skin surface of a user;

FIGS. 13A-13K are schematic views of a user interface generated by a downloadable software program; and

FIGS. 14A-14C are schematic view of different aspects of the user interface.

### DETAILED DESCRIPTION

Systems, methods and devices are provided for the non-invasive treatment stimulation of nerves to improve the general wellness and/or cognitive performance of a user. A procedure can be understood as being non-invasive when no break in the skin (or other surface of the body, such as a wound bed) is created through use of the method, and when there is no contact with an internal body cavity beyond a body orifice (e.g., beyond the mouth or beyond the external auditory meatus of the ear). In some ways, such non-invasive procedures can be distinguished from some invasive procedures (including minimally invasive procedures) in that the invasive procedures insert a substance or device into or through the skin (or other surface of the body, such as a wound bed) or into an internal body cavity beyond a body orifice.

In particular, the devices can transmit energy to, or in close proximity to, a selected nerve of the user in order to stimulate, block and/or modulate electrophysiological signals in that nerve. In some embodiments, one or more electrodes applied to the skin of the user generate currents within the tissue of the user. This may enable production and application of the electrical impulses so as to interact with the signals of one or more nerves, in order to achieve the therapeutic result.

In some embodiments, methods and devices are specifically designed for the treatment of a user by stimulation in or around a vagus nerve, with devices positioned non-invasively on or near a user's neck to target the cervical branch of the vagus nerve and/or in or around the auricular branch of the vagus nerve of the user (i.e., within the ear, on the surface of the ear, or on the user's head or upper neck near the auricular nerve). However, it will be recognized that some of the treatment paradigms described herein can be used with a variety of different vagal nerve stimulators, including implantable and/or percutaneous stimulation devices.

The methods and devices disclosed herein may be used for non-medical purposes, such as reducing stress and anxiety, enhancing relaxation, improving sleep, energy, concentration and mood, increasing mental or physical performance, promoting mental health, recovery and wellness and generally improving the health and wellbeing of a user.

In one example, Applicant conducted a focus study group to assess the effectiveness of the devices described herein to improve stress, anxiety, sleep, energy and mood. The participants conducted vagal nerve stimulation with the devices describes herein for 30 days. In the in-home focus group study. 34 participants were instructed to use the devices described herein twice a day for 30 days to assess the device's benefits and overall user experience. Self-assessment evaluations were reported after 7 and 30 days. After 30 days, users reported the following: 94% felt calmer, 91% felt their mood improved, 88% felt less anxious, 85% felt an improvement in stress, 82% felt more alert, 77% had more energy and 74% felt they sleep better.

The systems and methods described herein may also be configured to increase a user's cognitive performance, skill proficiency, judgement, vigilance, attention and memory by inducing neuroplasticity, improving neurobehavioral outcomes, enhancing focus and mitigating fatigue. Examples include enhancing learning and skill acquisition, such as second language learning vocabulary acquisition and/or enhancing cognitive performance in extreme environments, such as extreme stressors, multiday transoceanic operational and logistic flights, long duration remotely piloted aircraft missions and the like.

In other embodiments, the systems and methods are particularly useful for temporarily or permanently improving intelligence, learning capacity, memory retention, recall, mood, alertness and/or sleep efficiency in human beings. In certain embodiments, the methods and devices enhance neurostructural development over a period of time by increasing neurogenesis, neuronal plasticity and/or neural connectivity efficiency, and/or by improving the chemical microenvironment of the evolving neural network. In some embodiments, these enhancements may provide temporary improvement to enable an individual to, for example, accomplish a particular task while under duress, sleep deprivation, stress, anxiety or the like. In other embodiments, these changes permanently enhance the intelligence, emotional stability, and overall brain health of the treated individual.

In one example of the above embodiments, the systems and methods may be used to sense, assess and augment cognitive performance in operational environments, including reducing or mitigated stress/fatigue in extreme environments to augment performance. For example, the system and methods may increase cognitive performance in aerospace environments such as multi-day transoceanic operational and logistic flights as well as long duration remotely piloted aircraft missions. Fatigue resulting from these extreme stressors can evolve into chronic health problems, and cause decrements in judgement and vigilance resulting in severe aviation mishaps.

In another example, the system and methods may be used to improve second language learning. Applicant conducted a study at the Defense Language Institute (DLI) in Monterey, CA, the US Department of Defense's premier language school. The study was supported by Defense Advanced Research Projects Agency (DARPA)/AFRL within the DARPA Targeted Neuroplasticity Training (TNT) program. The study recruited 36 student participants from DLI's Arabic school house (nVNS=18 & Sham=18). Each subject was assessed on day 1 to establish a baseline. On days 2-4, two 2-minute stimulation treatments were self-administered to the vagus nerve by the subject, each before and after training. Assessments were taken each treatment day, and on day 5 where there was no treatment, assessments were conducted to assess possible carryover effects. The study showed a significant positive effect of the vagal nerve stimulation treatments over sham (p=0.025) on language recall, thereby suggesting the system described herein have the ability to significantly improve the recall of a foreign language compared to sham. The improvement on treatment on days 2-4 was maintained on day 5 demonstrating that the recall advantage that emerged during training was sustained after the completion of treatment.

The effectiveness of the neural network may be increased through neurogenesis, or the creation of more neurons in the brain. Alternatively, or in addition, and depending on the timing thereof within the framework of development, the effectiveness may be enhanced by increasing a connectivity of neurons within the brain of the individual and/or increasing the effective pruning of connections, or enhancing a neuronal plasticity within the brain of the individual. Neuronal plasticity is generally defined as the ability of the brain to change its structure and/or function in response to previous experience. It is essential for the establishment and refinement of neural networks during development and the formation of memory traces, the acquisition of specific skills and the storage of information.

In certain embodiments, the effectiveness of the neural network is increased sufficiently to temporarily or permanently improve the intelligence, learning capacity, memory retention, recall, mood, alertness and/or sleep efficiency in the individual. These enhancements may provide temporary improvement to enable an individual to, for example, accomplish a particular task while under duress, sleep deprivation or the like. In other embodiments, these changes permanently enhance the intelligence, emotional stability, and overall brain health of the treated individual.

In another aspect, systems and methods are providing for increasing the neurostructural development of a child to permanently increase the intelligence, learning capacity and/or memory retention of the child as the child grows and develops. The child may, for example, have an age of less than 18 years old, less than 10 years old, less than 5 years old or even less than 2 years old. In a related aspect, the child may still be within the womb, and the modulation of a maternal inflammation state may be desirable. A more complete description of these embodiments can be found in U.S. patent application Ser. No. 17/844,368, filed Jun. 21, 2022, the complete disclosure of which is incorporated herein by reference for all purposes.

In other embodiments, the systems and methods are useful for directly or indirectly increasing an activity of telomerase within the user. Increasing telomerase activity protects the end of the chromosome from DNA damage or from fusion with neighboring chromosomes, thereby maintaining the length of the telomeres or at least inhibiting the natural reduction of telomeres. Inhibiting the reduction of telomere lengths may potentially increase the lifespan or health span on an individual. Lifespan as defined herein as the duration of life of an individual. Health span is defined herein as the period of one's life that one is substantially free from serious disease. A disease is “serious” if it is a leading cause of death, which includes heart disease, lung, colorectal, breast or prostate cancer, COPD, stroke, lower respiratory infections, Alzheimer's disease and Type 2 diabetes. A more complete description of these embodiments can be found in U.S. patent application Ser. No. 17/731,393, filed Apr. 28, 2022, the complete disclosure of which is incorporated herein by reference for all purposes.

In other embodiments, the systems and methods are useful for treating user's suffering from systemic inflammation or chronic stress, anxiety or other disorders that invoke a chronic stress response resulting from elevated cortisol levels. These elevated cortisol levels may contribute to accelerated shortening of telomere lengths in cellular DNA, such as chronic pain, tumors or other disorders of the pituitary gland, depression, mood disorders, fear, perceived threats to safety, status or well-being, fatigue, irritability, headache, intestinal problems, increased blood pressure, poor sleep and the like. Reduction of these telomere lengths may potentially shorten the overall longevity and/or health span of the user.

Cortisol is a potent anti-inflammatory that functions to mobilize glucose reserves for energy and modulate inflammation. Cortisol also may facilitate the consolidation of fear-based memories for future survival and avoidance of danger. Although short-term stress may be adaptive, maladaptive responses (e.g., magnification, rumination, helplessness) to pain or non-pain-related stressors may intensify cortisol secretion and condition a sensitized physiologic stress response that is readily recruited. Ultimately, a prolonged or exaggerated stress response may perpetuate cortisol dysfunction, widespread inflammation, and pain. Stress may be unavoidable in life, and challenges are inherent to success; however, humans have the capability to modify what they perceive as stressful and how they respond to it. Exaggerated psychological responses (ego, catastrophizing) following maladaptive cognitive appraisals of potential stressors as threatening may exacerbate cortisol secretion and facilitate the consolidation of fear-based memories of pain or non-pain-related stressors; however, coping, cognitive reappraisal, or confrontation of stressors may minimize cortisol secretion and prevent chronic, recurrent pain.

Studies have shown that heightened cortisol responsivity to psychological stress is associated with accelerated cellular aging as indexed by leukocyte telomere length. This indicates that heightened cortisol responsivity is not simply a consequence of more advanced cellular aging but may contribute to the cellular aging process. Cortisol also suppresses telomerase activation in immune system cells so that telomeres are no longer protected during cell division and become progressively shorter. This leads to early cell aging and distorted replicas of the original cell that could lead to cancer and other diseases.

Applying vagal nerve stimulation to a user with a specific treatment protocols described herein may reduce the level of circulating cortisol in the user. This reduction in chronic cortisol levels may alter the chronic stress response, alleviating the many symptoms associated with such response. In addition, vagal nerve stimulation with the specific stimulation methods and devices described herein may increase an activity of telomerase within the user. Increasing telomerase activity protects the end of the chromosome from DNA damage or from fusion with neighboring chromosomes, thereby maintaining the length of the telomeres or at least inhibiting the natural reduction of telomeres.

In certain embodiments, the electrical impulse and the stimulation protocol are sufficient to modulate the vagus nerve to reduce a cortisol level within the user, particularly the amount of circulating cortisol within the user. Thus, devices and methods provided herein increase the ability of the parasympathetic nerve to adapt to upward regulation and stress. This not only provides a mechanism for stress control, but mitigates the impact of chronic stress by reducing the levels of circulating cortisol. Reducing these levels of cortisol deaccelerates telomere shortening and thus the ageing process. A more complete description of these embodiments can be found in U.S. patent application Ser. No. 17/844,368, filed Jun. 21, 2022, the complete disclosure of which is incorporated herein by reference for all purposes.

In some embodiments, various methods can use vagal nerve stimulation to suppress inflammation, thereby increasing the effectiveness of telomerase to maintain telomere lengths in cellular DNA. In some embodiments, some methods and devices involve the inhibition of pro-inflammatory cytokines, or more specifically, stimulation of the vagus nerve to inhibit and/or block the release of such pro-inflammatory cytokines. In some embodiments, some methods and devices use vagal nerve stimulation to increase the concentration or effectiveness of anti-inflammatory cytokines. A more complete description of these embodiments can be found in U.S. patent application Ser. No. 17/731,393, filed Apr. 28, 2022, the complete disclosure of which is incorporated herein by reference for all purposes.

The methods and devices disclosed herein can be used to prevent, diagnose, monitor, ameliorate, or treat a medical condition, a disease, or a disorder of a user, such as a mammal, such as an animal, such as a human, whether male or female, whether infant, child, adult, or elderly, or others.

For example, the devices can be configured to prevent, diagnose, monitor, ameliorate, or treat a neurological condition, such as epilepsy, headache, whether primary headaches, such as cluster, migraine or tension, or secondary headaches, caused by, for example, acute sinusitis, arterial tears, blood clots, aneurysms, glaucoma, tumors, medication overuse headaches, thunderclap headaches, concussion (e.g., post-concussion syndrome), trigeminal neuralgia and the like, seizures, vertigo, dizziness, aneurysm, palsy, Parkinson's disease, Alzheimer's disease, post-traumatic stress disorder (PTSD) or others, as understood to skilled artisans and which are only omitted here for brevity.

For example, the medical devices can be configured to prevent, diagnose, monitor, ameliorate, or treat conditions associated with replicating pathogens. The replicating pathogen may include a bacteria, fungi, protozoa, worm, infectious protein (e.g., prion) or a virus, such as an RNA virus. In one particular embodiment, the virus comprises a virus that contains a sensitizing and/or allergenic protein or other molecule that triggers an allergic or inflammatory response in the user, such as a virus in the coronaviridae or coronavirus family (e.g., COVID 19). The methods and systems of the present invention reduce the expression of inflammatory mediators that are elevated in ARDS and other inflammatory disorders, thereby ameliorating the overactivity of the immune reaction in user's suffering from certain disorders associated with replicating pathogen. This therapy provides potent anti-inflammatory activity without the negative side effect of conventional immune suppression techniques and drugs, such as steroids. In addition, the methods and systems of the present invention decrease the magnitude of constriction of bronchial smooth muscle, thereby improving the user's breathing in situations involving shortness of breath and impaired oxygen saturation, such as ARDS caused by certain replicating pathogens (e.g., COVID 19). A more complete description of these embodiments can be found in U.S. patent application Ser. No. 16,838,953, filed Apr. 2, 2020 and Ser. No. 17/472,962, filed Sep. 10, 2021, the complete disclosure of which is incorporated herein by reference for all purposes.

For example, the medical devices can be configured to prevent, diagnose, monitor, ameliorate, avert or treat a stroke and/or transient ischemic attack, to ameliorate or limit the effects of an acute stroke or transient ischemic attack, and/or to rehabilitate a stroke user.

For example, the medical devices can be configured to prevent, diagnose, monitor, ameliorate, or treat neurological, neuropsychological, or neuropsychiatric activity, such as a modulation of neuronal function or processing to affect a functional outcome. The modulation of neuronal function can be useful with regard to diagnosing, monitoring, preventing, treating, or ameliorating neurological, psychiatric, psychological, conscious state, behavioral, mood, or thought activity. For example, this activity can manifests itself in a form of a disorder, such as attention or cognitive disorders (e.g., Autistic Spectrum Disorders), mood disorder (e.g., major depressive disorder, bipolar disorder, dysthymic disorder), anxiety disorder (e.g., panic disorder, posttraumatic stress disorder, obsessive-compulsive disorder, phobic disorder); neurodegenerative diseases (e.g., multiple sclerosis, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), movement disorders (e.g., dyskinesia, tremor, dystonia, chorea and ballism, tic syndromes, Tourette's Syndrome, myoclonus, drug-induced movement disorders, Wilson's Disease, Paroxysmal Dyskinesias, Stiff Man Syndrome and Akinetic-Rigid Syndromes and Parkinsonism), epilepsy, tinnitus, pain, phantom pain, diabetes neuropathy, enhancing or diminishing any neurological or psychiatric function not just an abnormality or disorder or others, as understood to skilled artisans and which are only omitted here for brevity. Neurological activity that may be modulated can include normal functions, such as alertness, conscious state, drive, fear, anger, anxiety, repetitive behavior, impulses, urges, obsessions, euphoria, sadness, and the fight or flight response, as well as instability, vertigo, dizziness, fatigue, photophobia, concentration dysfunction, memory disorders, headache, dizziness, irritability, fatigue, visual disturbances, sensitivity to noise (misophonia, hyperacusis, phonophobia), judgment problems, depression, symptoms of traumatic brain injury (whether physical, emotional, social, or chemical), autonomic functions, which includes sympathetic or parasympathetic functions (e.g., control of heart rate), somatic functions, or enteric functions.

For example, some systems and methods can be configured to prevent, diagnose, monitor, ameliorate, or treat an inflammatory disease or disorder, such as Alzheimer's disease, ankylosing spondylitis, arthritis (osteoarthritis, rheumatoid arthritis (RA), Sjôgren's syndrome, temporal arteritis, Type 2 diabetes, psoriatic arthritis, asthma, atherosclerosis, Crohn's disease, colitis, dermatitis, diverticulitis, fibromyalgia, hepatitis, irritable bowel syndrome (IBS), systemic lupus erythematous (SLE), nephritis, fibromyalgia, Celiac disease, Parkinson's disease, ulcerative colitis, chronic peptic ulcer, tuberculosis, periodontitis, sinusitis, hepatitis, Grave's disease, psoriasis, pernicious anemia (PA), peripheral neuropathy, lupus or others, as understood to skilled artisans and which are only omitted here for brevity. For example, some systems and methods can be configured to prevent, diagnose, monitor, ameliorate, or treat a gastrointestinal condition, such as ileus, irritable bowel syndrome, Crohn's disease, ulcerative colitis, diverticulitis, gastroesophageal reflux disease, or others, as understood to skilled artisans and which are only omitted here for brevity

For example, the devices described herein can be configured to prevent, diagnose, monitor, ameliorate, or treat a gastrointestinal condition, such as post-operative ileus (POI), irritable bowel syndrome, Crohn's disease, ulcerative colitis, diverticulitis, gastroesophageal reflux disease, or others, as understood to skilled artisans and which are only omitted here for brevity.

For example, the devices described herein can be configured to prevent, diagnose, monitor, ameliorate, or treat a bronchial disorder, such as asthma, bronchitis, pneumonia, COPD, or others, as understood to skilled artisans and which are only omitted here for brevity.

For example, the devices described herein can be configured to prevent, diagnose, monitor, ameliorate, or treat a coronary artery disease, heart attack, arrhythmia, cardiomyopathy, or others, as understood to skilled artisans and which are only omitted here for brevity.

For example, the devices described herein can be configured to prevent, diagnose, monitor, ameliorate, or treat a urinary disorder, such as urinary incontinence, urinalysis, overactive bladder, or others, as understood to skilled artisans and which are only omitted here for brevity.

For example, the devices described herein can be configured to prevent, diagnose, monitor, ameliorate, or treat addiction, alcoholism, alcohol dependence or opioid-use disorders. In some cases, the devices may be used as a bridge-therapy for alcohol dependence or opioid-use disorders to reduce opioid or alcohol cravings sufficiently to remain drug-free prior to receiving further treatment, such as prescription injection medications or other alcohol and drug recovery programs.

For example, the devices described herein can be configured to prevent, diagnose, monitor, ameliorate, or treat a cancer, such as bladder cancer, breast cancer, prostate cancer, lung cancer, colon or rectal cancer, skin cancer, thyroid cancer, brain cancer, leukemia, liver cancer, lymphoma, pancreatic cancer, or others, as understood to skilled artisans and which are only omitted here for brevity.

For example, the devices described herein can be configured to prevent, diagnose, monitor, ameliorate, or treat a metabolic disorder, such as diabetes (type 1, type 2, or gestational), Gaucher's disease, sick cell anemia, cystic fibrosis, hemochromatosis, or others, as understood to skilled artisans and which are only omitted here for brevity.

For example, the neurostimulator can modulate central or peripheral nervous systems. For example, the neurostimulator can be enable spinal cord stimulation to provide therapy for intractable pain and refractory angina; occipital nerve stimulation to provide therapy for occipital neuralgia and transformed migraine; afferent vagus nerve modulation to provide therapy for a host of neurological and neuropsychiatric disorders, such as epilepsy, depression, Parkinson's disease, bulemia, anxiety/obsessive compulsive disorders, Alzheimer's disease, autism, and neurogenic pain; efferent vagus nerve stimulation for rate control in atrial fibrillation, and to provide therapy for congestive heart failure; gastric nerves or gastric wall stimulation to provide therapy for obesity; sacral nerve stimulation to provide therapy for urinary urge incontinence; deep brain stimulation to provide therapy for Parkinson's disease, and other neurological and neuropsychiatric disorders; cavernous nerve stimulation to provide therapy for erectile dysfunction.

In other embodiments, the stimulator can be enable spinal cord stimulation to provide therapy for intractable pain and refractory angina; occipital nerve stimulation to provide therapy for occipital neuralgia and transformed migraine; afferent vagus nerve modulation to provide therapy for a host of neurological and neuropsychiatric disorders, such as epilepsy, depression, Parkinson's disease, bulemia, anxiety/obsessive compulsive disorders, Alzheimer's disease, autism, and neurogenic pain; efferent vagus nerve stimulation for rate control in atrial fibrillation, and to provide therapy for congestive heart failure; gastric nerves or gastric wall stimulation to provide therapy for obesity; sacral nerve stimulation to provide therapy for urinary urge incontinence; deep brain stimulation to provide therapy for Parkinson's disease, and other neurological and neuropsychiatric disorders; cavernous nerve stimulation to provide therapy for erectile dysfunction.

In other embodiments, the stimulator can be used to provide therapy to user's suffering from post-operative symptoms following major surgery and/or for treating users in critical or intensive care. Post-operative symptoms may include nausea, vomiting, constipation, gas, post-operative ileus, post-operative pain, restlessness, sleeplessness and the like. In some embodiments, the systems and methods are particularly useful for mitigating or eliminating the impairment of gastrointestinal (GI) motility after intra-abdominal or nonabdominal surgery, such as post-operative ileus (POI). In some embodiments, the systems and methods may reduce opioid-induced and/or inflammatory-induced dysfunction of intestinal transit to improve motility, thereby allowing faster release of user's from the hospital.

A more complete description of some of the applications for non-invasive nerve stimulation can be found in U.S. Pat. Nos. 9,037,247, 8,972,004, 8,868,177, 9,089,719, 8,874,205, 8,676,330, 10,279,163, 8,874,227, 9,174,066, 9,566,426, 9,174,045, 10,252,074, 9,126,050, 11,517,742, 10,232,178, 9,403,001, 9,174,049, 10,512,769, 11,432,760, 10,537,728, 11,590,341 and 11,027,127, the complete disclosures of which are incorporated herein by reference.

The fact that electrical stimulation of a vagus nerve can be used to treat many disorders and conditions may be understood as follows. The vagus nerve is composed of motor and sensory fibers. The vagus nerve leaves the cranium, passes down the neck within the carotid sheath to the root of the neck, then passes to the chest and abdomen, where it contributes to the innervation of the viscera. A human vagus nerve (tenth cranial nerve, paired left and right) comprises of over 100,000 nerve fibers (axons), mostly organized into groups. The groups are contained within fascicles of varying sizes, which branch and converge along the nerve. Under normal physiological conditions, each fiber conducts electrical impulses only in one direction, which is defined to be the orthodromic direction, and which is opposite the antidromic direction. However, external electrical stimulation of the nerve may produce action potentials that propagate in orthodromic and antidromic directions. Besides efferent output fibers that convey signals to the various organs in the body from the central nervous system, the vagus nerve conveys sensory (afferent) information about the state of the body's organs back to the central nervous system. Some 80-90% of the nerve fibers in the vagus nerve are afferent (sensory) nerves, communicating the state of the viscera to the central nervous system.

The largest nerve fibers within a left or right vagus nerve are approximately 20 μm in diameter and are heavily myelinated, whereas only the smallest nerve fibers of less than about 1 μm in diameter are completely unmyelinated. When the distal part of a nerve is electrically stimulated, a compound action potential may be recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories, with approximate diameters as follows: A-alpha fibers (afferent or efferent fibers, 12-20 μm diameter), A-beta fibers (afferent or efferent fibers, 5-12 μm), A-gamma fibers (efferent fibers, 3-7 μm), A-delta fibers (afferent fibers, 2-5 μm), B fibers (1-3 μm) and C fibers (unmyelinated, 0.4-1.2 μm). The diameters of group A and group B fibers include the thickness of the myelin sheaths.

The vagus (or vagal) afferent nerve fibers arise from cell bodies located in the vagal sensory ganglia, which take the form of swellings near the base of the skull. Vagal afferents traverse the brainstem in the solitary tract, with some eighty percent of the terminating synapses being located in the nucleus of the tractus solitarius (or nucleus tractus solitarii, nucleus tractus solitarius, or NTS). The NTS projects to a wide variety of structures in the central nervous system, such as the amygdala, raphe nuclei, periaqueductal gray, nucleus paragigantocellurlais, olfactory tubercule, locus ceruleus, nucleus ambiguous and the hypothalamus. The NTS also projects to the parabrachial nucleus, which in turn projects to the hypothalamus, the thalamus, the amygdala, the anterior insula, and infralimbic cortex, lateral prefrontal cortex, and other cortical regions [JEAN A. The nucleus tractus solitarius: neuroanatomic, neurochemical and functional aspects. Arch Int Physiol Biochim Biophys 99 (5, 1991): A3-A52 the disclosure of which is incorporated herein by reference for all purposes as if copied and pasted herein]. Thus, stimulation of vagal afferents can modulate the activity of many structures of the brain and brainstem through these projections.

With regard to vagal efferent nerve fibers, two vagal components have evolved in the brainstem to regulate peripheral parasympathetic functions. The dorsal vagal complex, consisting of the dorsal motor nucleus and its connections controls parasympathetic function primarily below the level of the diaphragm, while the ventral vagal complex, comprised of nucleus ambiguous and nucleus retrofacial, controls functions primarily above the diaphragm in organs such as the heart, thymus and lungs, as well as other glands and tissues of the neck and upper chest, and specialized muscles such as those of the esophageal complex. For example, the cell bodies for the preganglionic parasympathetic vagal neurons that innervate the heart reside in the nucleus ambiguus, which is relevant to potential cardiovascular side effects that may be produced by vagus nerve stimulation.

The vagus efferent fibers innervate parasympathetic ganglionic neurons that are located in or adjacent to each target organ. The vagal parasympathetic tone resulting from the activity of these fibers is balanced reflexively in part by sympathetic innervations. Consequently, electrical stimulation of a vagus nerve may result not only in modulation of parasympathetic activity in postganglionic nerve fibers, but also a reflex modulation of sympathetic activity. The ability of a vagus nerve to bring about widespread changes in autonomic activity, either directly through modulation of vagal efferent nerves, or indirectly via activation of brainstem and brain functions that are brought about by electrical stimulation of vagal afferent nerves, accounts for the fact that vagus nerve stimulation can treat many different medical conditions in many end organs. Selective treatment of particular conditions is possible because the parameters of the electrical stimulation (e.g. frequency, amplitude, pulse width, etc.) may selectively activate or modulate the activity of particular afferent or efferent A, B, and/or C fibers that result in a particular physiological response in each individual.

### Treatment Paradigms

Depending on the medical indication, whether it is a chronic or acute treatment, and the natural history of the disease, different treatment protocols may be used. In particular, applicant has discovered that it is not necessary to “continuously stimulate” the vagus nerve or to in order to provide efficacious benefits to users. The term “continuously stimulate” as defined herein means stimulation that follows a certain On/Off pattern continuously 24 hours/day. For example, existing implantable vagal nerve stimulators “continuously stimulate” the vagus nerve with a pattern of 30 seconds ON/5 minutes OFF (or the like) for 24 hours/day and seven days/week. Applicant has determined that this continuous stimulation is not necessary to provide the desired benefits of the device.

The present description contemplates at least three types of interventions involving stimulation of a vagus nerve: prophylactic, acute and compensatory (rehabilitative). Among these, the acute treatment involves the fewest administrations of vagus nerve stimulations, which begin upon the appearance of symptoms. It is intended primarily to enlist and engage the autonomic nervous system to inhibit excitatory neurotransmissions that accompany the symptoms. The prophylactic treatment resembles the acute treatment in the sense that it is administered as though acute symptoms had just occurred (even though they have not) and is repeated at regular intervals, as though the symptoms were reoccurring (even though they are not). The rehabilitative or compensatory treatments, on the other hand, seek to promote long-term adjustments in the central nervous system, compensating for deficiencies that arose as the result of the user's disease by making new neural circuits.

A vagus nerve stimulation treatment is conducted for continuous period of thirty seconds to five minutes, preferably about 90 seconds to about three minutes and more preferably about two minutes (each defined as a single dose). After a dose has been completed, the therapy is stopped for a period of time (depending on the treatment as described below). For prophylactic treatments, such as a treatment to reduce cortisol or other stress hormone levels, the therapy preferably comprises multiple doses/day over a period of time that may last from one day to a number of months or even years. In certain embodiments, the treatment will comprise multiple doses at predetermined times during the day and/or at predetermined intervals throughout the day. In exemplary embodiments, the treatment comprises one of the following: (1) 1-6, preferably 2-3 doses/day at predetermined intervals or times; (2) two doses, either consecutively, or separated by 5 min at predetermined intervals or times, preferably one to three times/day; (3) 3 doses, either consecutively or separated by 5 min again at predetermined intervals or times, such as 2 or 3 times/day; or (4) 1-3 doses, either consecutively or separated by 5 min, 4-6 times per day.

In exemplary embodiments, the treatment comprises one of the following: (1) 2-12 doses/day, preferably about 2-4 doses, at predetermined intervals or times; (2) two doses, either consecutively, or separated by 5 min at predetermined intervals or times, preferably two to four times/day; (3) 3 doses, either consecutively or separated by 5 min again at predetermined intervals or times, such as 2 or 3 times/day; or (4) 1-3 doses, either consecutively or separated by 5 min, 4-6 times per day.

For certain treatments, the time of day can be more important than the time interval between treatments. For example, the locus correleus has periods of time during a 24 hour day wherein it has inactive periods and active periods. Typically, the inactive periods can occur in the late afternoon or in the middle of the night when the user is asleep. It is during the inactive periods that the levels of inhibitory neurotransmitters in the brain that are generated by the locus correleus are reduced. This may have an impact on certain applications.

In these embodiments, the prophylactic treatment may comprise multiple doses/day timed for periods of inactivity of the locus correleus. In one embodiment, a treatment comprises one or more doses administered 2-3 times per day or 2-3 “treatment sessions” per day. The treatment sessions preferably occur during the late afternoon or late evening, in the middle of the night and again in the morning when the user wakes up. In an exemplary embodiment, each treatment session comprises 1˜4 doses, preferably 2-3 doses, with each dose lasting for about 60 seconds to about 5 minutes, preferably about 90 seconds to about three minutes.

For other applications, the intervals between treatment sessions may be the most important as applicant has determined that stimulation of the vagus nerve can have a prolonged effect on the inhibitor neurotransmitters levels in the brain, e.g., at least one hour, up to 3 hours and sometimes up to 8 hours. In one embodiment, a treatment comprises one or more doses (i.e., treatment sessions) administered at intervals during a 24 hour period. In a preferred embodiment, there are 1-5 such treatment sessions, preferably 2-4 treatment sessions. Each treatment session preferably comprises 1-3 doses, each “dose” lasting between about 60 seconds to about five minutes, preferably about 90 seconds to about 150 seconds, more preferably about 2 minutes.

For all of the treatments listed above, one may alternate treatment between left and right sides, or one may treat ipsilateral or contralateral to one side of the brain, respectively. Or for a single treatment, one may treat one minute on one side followed by one minute on the opposite side. Variations of these treatment paradigms may be chosen on a user-by-user basis. However, it is understood that parameters of the stimulation protocol may be varied in response to heterogeneity in the symptoms of users. Different stimulation parameters may also be selected as the course of the user's condition changes. In preferred embodiments, the disclosed methods and devices do not produce clinically significant side effects, such as agitation or anxiety, or changes in heart rate or blood pressure. A more complete description of some of these embodiments can be found in U.S. Pat. No. 10,441,780, the complete disclosure of which is incorporated herein by reference.

### Description of Various Nerve Stimulating/Modulating Devices

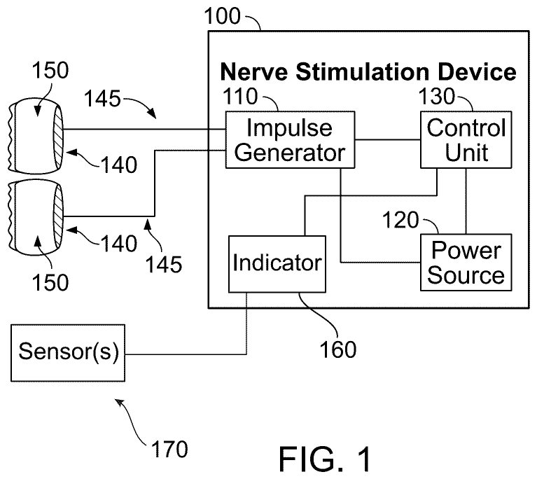

Referring now to FIG. 1, an electrode-based nerve stimulating and/or modulating device 100 is provided for delivering impulses of energy to nerves. As shown, device 100 may include an impulse or signal generator 110, an energy or power source 120 coupled to the impulse generator 110 and/or a control unit 130 in communication with the impulse generator 110 and coupled to the energy source 120. Device 100 further includes one or more electrodes 140 coupled via wires 145 (or wirelessly) to impulse generator 110. Alternatively, electrodes 140 may be housed within, or on the outer surface of, device 100.

In some embodiments, the same impulse generator 110, energy source 120, and control unit 130 may be used for either a magnetic stimulator or an electrode-based stimulator, allowing the user to change parameter settings depending on whether magnetic coils or the electrodes 140 are attached.

Although a pair of electrodes 140 is shown in FIG. 1, in practice the electrodes may also comprise one electrode, or three or more distinct electrode elements, each of which is connected in series or in parallel to the impulse generator 110. Thus, the electrodes 140 that are shown in FIG. 1 represent some, most, many, or all electrodes of the device collectively.

Electrodes 140 may include a suitable adhesive that secured them to a skin surface. Suitable adhesive electrodes for use herein may include electrode pads, self-adhesive electrodes or the like. In this embodiment, electrodes 140 may be placed in a suitable location on the user's neck and adhered thereto. Electrodes 140 receive electrical impulses from pulse generator 110. The duration, amplitude, frequency and treatment paradigm for the electrical impulses may be controlled by controller 130, a mobile device, a remote computer, processor or server, or via another electronic device coupled to pulse generator 110. Suitable mobile devices include a wearable computing devices, such as a smartwatch, Whoop®, Fitbit®, Garmin® or the like, a mobile phone, a mobile processing device (e.g., laptop computers or tablets) and the like. This embodiment allows, for example, a physician to secure electrodes 140 to the user's neck such that the treatment paradigm may be followed without user involvement. This is particularly useful for treating users that are unable or unwilling to self-treat. For example, in some cases, users recovering from surgery, such as major colorectal surgery may be either incapable of self-treatment, or their compliance with the treatment protocol may not be complete. In another example, older users may not have suitable mental faculties for self-treatment.

Stimulator 100, or certain components of stimulator, may be housed in an outer housing, or a covering or patch 330 to protect stimulator from the environment. The patch may include a suitable adhesive strip or pad on one surface for adhering the patch and stimulator to the outer skin surface of the user.

The stimulator in this embodiment includes one or more electrodes. The stimulator may also include a power source such as a battery, and a signal generator for applying the electrical impulses to the electrodes. In one such embodiment, the power source, e.g., a battery, is disposed within the patch and coupled to the electrodes. In another embodiment, the signal generator includes flexible circuitry within the patch and coupled to the energy source and the electrodes. Alternatively, the power source and/or the signal generator may be remote from the patch and wirelessly coupled, or directly connected to the electrodes, as discussed above. An external controller may be wirelessly coupled to the stimulator to provide a stimulation protocol to the signal generator and to control other key functions of the signal, such as power, amplitude, duration frequency and the like.

The stimulator may reside in a housing that is removably coupled to the patch via a snap-fitting, Velcro, or other suitable attachment means. In this embodiment, the patch may be adhered to the user and the stimulator may be removed and reattached without removing the patch. This allows the healthcare professions to, for example, recharge the battery, troubleshoot the device and/or control the stimulation therapy on the device.

The stimulator may also include a conductive fluid, such as a gel pad, disposed between the electrode(s) and the user's outer skin surface to enhance conductivity of the electrical impulses through the outer skin surface to the nerve.

Alternatively, the outer covering may comprise any wearable material that may include the stimulator. For example, depending on the location of the target nerve on the user's body, the stimulator may be attached to, or embedded within, a wearable garment, such as a shirt, scarf, watch, hat, gloves, pants, shoes, boots, socks, underwear, belt, dress, jacket, sweater, ear muffs, or the like. The wearable garment may also comprise an accessory, such as a wristband, ankle or wrist bracelet, necklace, earrings, a compression garment, an ankle or knee brace or the like.

In yet another embodiment, the garment itself is the stimulator. For example, the garment may comprise an electronic textile or e-textile that includes fabrics that enable digital components, such as electrodes, pulse generators, batteries wireless receivers and other electronic components to be embedded therein. Electronic textiles are distinct from wearable garments because the emphasis is placed on the seamless integration of textiles with electronic elements like microcontrollers, sensors, and actuators. In one embodiment, the electronic textile may comprise an organic electronics material that is conducting and has insulated electrical components that allows the garment to be washed without damaging the electronic components.

The stimulator may also include an array of electrodes. The electrode array may include multiple sets of electrodes with each set of electrodes configured to apply electrical impulses through the outer skin surface of the user, as discussed above. Each of the sets of electrodes may be individually coupled to the pulse generator, either directly, through wires, or wireless as described above. The electrode array may have multiple patterns. For example, the array may be linear, square, circular or any other suitable shape.

In certain embodiments, the electrode array comprises two or more sets of electrodes, each spaced apart from each other between about 2 mm to about 25 mm, preferably between about 4 mm to about 10 mm. The electrode array preferably comprises a shape that substantially corresponds to a target area of the user's neck. In one embodiment, the target area is the area on the neck that allows for electrical impulses to be passed through the skin to the vagus nerve (discussed in detail below).

The electrode sets may each be individually coupled to the pulse generator and/or the controller such that electrical impulses can be applied to all of the electrode sets, some of the electrode sets or only one of the electrode sets. In certain embodiments, the controller is configured to apply electrode impulses to only those electrodes positioned optimally for stimulating the nerve. In addition, the selection of electrodes may be dynamic and change over time.

In one such embodiment, the electrodes are arranged in an array or matrix that may contain tens to hundreds of microelectrodes. The microelectrodes may each be independently coupled to the pulse generator 110 such that the pulse generator can apply current to any one or a plurality of the microelectrodes. In some embodiments, groups of the microelectrodes are coupled together and then coupled to the pulse generator 110 such that electric current can be applied independently to each group. In an exemplary embodiment, the electrodes have a size of about 0.5 to 2.0 mm, preferably about 1.0 mm, and are spaced from each other a distance of about 0.5 to about 10 mm, preferably between about 2.0 mm and 5.0 mm (e.g., 3.0 mm).

Stimulator 100 further comprises one or more sensors 170 coupled to stimulator 100 and/or electrodes 140 (or the microelectrodes in the array) for detecting whether the nerve has been stimulated, the amplitude of the stimulation, or whether the nerve has been stimulated with sufficient amplitude and other parameters to fire an action potential. The sensors 170 may detect a physiological parameter of the user. Physiological parameters may include, for example, blood flow associated with a nerve, such as vagal artery or cerebral blood flow, heart rate or variability, ECG, respiration status, depth and rate, core temperature, hydration, blood pressure, brain function, oxygenation, skin impedance, and skin temperature, pupil diameter (e.g., pupil dilation), galvanic skin response, selected biomarkers or other chemicals, a property of a voice of the user, a laryngeal electromyographic signal, an electroglottographic signal, a property of the autonomic nervous system and the like. Alternatively, the sensors 170 may be coupled to the electrodes 140 and may sense one or more parameters of the electrodes, such as impedance, amplitude, voltage or the like.

The sensors 170 may also be coupled to the controller 130. In this embodiment, the controller 130 is configured to receive input from the sensors and to direct the pulse generator 110 to apply electrical impulses to one or more sets of the electrodes 140 based on this input. For example, the sensors 170 may provide data that suggests that one or more of the sets of electrodes is not positioned properly to stimulate the nerve, or to stimulate the nerve at the optimal signal strength to cause the nerve to fire an action potential. The controller 130 is configured to shift the electrical impulse to the set or sets of electrodes that provide a sufficient electrical impulse to the nerve to cause it to fire an action potential. In this manner, the controller 130 can optimize the application of the electrical impulses to the nerve.

Sensor(s) 170 may be coupled to electrodes 140, or they may be formed as part of the electrodes 140. Alternatively, sensor(s) 140 may be only coupled to stimulator 100, or they may be coupled to a separate device, such as a mobile device (discussed below). In certain embodiments, stimulator 100 will comprise a housing that includes both electrodes 140 and sensors 170, as discussed in more detail below.

In certain embodiments, sensor(s) 170 are configured to detect a target position for stimulating a selected nerve within a user. The target position may, for example, be located on an outer skin surface of the user and the selected nerve may be located within the user under the skin surface. In some cases, the selected nerve may be located deep within the user, i.e., greater than about 5 mm below the outer skin surface, greater than about 10 mm, or even greater than about 20 mm below. In one such embodiment, the selected nerve is the vagus nerve and the target location is a position on the outer skin surface of the neck and/or the ear of the user suitable for passing an electrical impulse through the skin sufficient to modulate the vagus nerve.

In one embodiment, sensor 170 comprises a heart pulse sensor configured to detect a heart pulse in the user. The heart pulse sensor may be any suitable sensor known in the art for detecting the heart pulse of a user, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 170 is located adjacent to, or near, the user's heart pulse.

The heart pulse sensor is preferably designed to contact the user's outer skin surface and detect a pulse adjacent to, or near the sensor. However, in certain embodiments, the heart pulse sensor may be designed to detect the heart pulse without contacting the skin surface, e.g., through vibration, sound or other detection mechanisms. In these embodiments, sensor 170 may, for example, be located within stimulator 100, or within a separate device.

Sensors 170 may be coupled to an indicator 160 within stimulator 100, or within a separate device, such as a mobile device (discussed in more detail below). Indicator 160 is configured to generate an alert when sensors 170 have detected the target nerve. The alert may be, for example, a visual, tactile or audial alert, that provides the user with an indication that the sensor 170 has detected the target location.

In one such embodiment, sensor 170 comprises a heart pulse sensor that is configured to detect a heart pulse emanating from a blood vessel in the user, such as the carotid artery in the user's neck, the temporal artery at the temple above and to the outer side of the eye, the radial artery in the user's wrist, the elbow of the top of the foot. In an exemplary embodiment, the heart pulse sensor is configured to detect a heart pulse in the carotid artery. The vagus nerve is situated within the carotid sheath, near the carotid artery and the interior jugular vein. The carotid sheath is located at the lateral boundary of the retropharyngeal space on each side of the neck and deep to the sternocleidomastoideole. A more complete description of some of these embodiments can be found in U.S. patent application Ser. No. 17/744,557, filed May 13, 2022, the complete disclosure of which is incorporated herein by reference for all purposes.

The three major structures within the carotid sheath are the common carotid artery, the internal jugular vein and the vagus nerve. The carotid artery lies medial to the internal jugular vein, and the vagus nerve is situated posteriorly between the two vessels. Proceeding from the skin of the neck above the sternocleidomastoideole to the vagus nerve, a line may pass successively through the sternocleidomastoideole, the carotid sheath and the internal jugular vein, unless the position on the skin is immediately to either side of the external jugular vein. In the latter case, the line may pass successively through only the sternocleidomastoideole and the carotid sheath before encountering the vagus nerve, missing the interior jugular vein. Accordingly, a point on the neck adjacent to the external jugular vein might be preferred for non-invasive stimulation of the vagus nerve.

Sensors 170 are configured to detect the heart pulse emanating from the carotid artery to provide an indication that electrodes 150 are located adjacent to, or near the carotid sheath and/or the external jugular vein and thus near the vagus nerve. This provides confirmation to the user that the device is positioned optimally for stimulating the vagus nerve.

In certain embodiments, sensors 170 may be configured to detect a magnitude of the heart pulse emanating from the carotid artery. In these embodiments, the sensors 170 may be configured, for example, to only provide an indication that the heart pulse has been detected when the magnitude of heart pulse reaches a threshold level, indicating that the sensor is close to the carotid artery. Alternatively, the sensors 170 may transmit the magnitude of heart pulse detected to a controller or suitable electronics within stimulator, or a separate mobile device.

In certain embodiments, indicator 160 is configured to transmit an alert that is associated with the magnitude of the heart pulse. For example, the alert may comprise an audible sound that increases in decibel level as the magnitude increase. In another example, the alert may comprise a vibration that increases in intensity or frequency as the magnitude of the heart pulse increases. In yet another example, the alert may comprise a visual signal, such as a blinking light that increases in intensity with heart pulse magnitude, different colored lights associated with threshold magnitudes of heart pulse, or another visual signal, such as bars, lines or other shapes that increase in size (e.g., length or width) with increasing heart pulse magnitude.

The indicator 160 may further be configured to provide a second alert when the magnitude of the heart pulse reaches a threshold level associated with optimal positioning of the sensor 170 and/or the electrodes 150. For example, if the indicator is providing a blinking light that increases in intensity with heart pulse magnitude, the second alert may be that the blinking light stops blinking and becomes constant, or it changes color (e.g., from yellow to green), or a separate alert is produced, such as a sound, vibration or the like.

In this embodiment, the sensor 160 may comprise a heart pulse sensor configured to contact the outer skin surface of the user and directly detect the pulse within the carotid sheath, such as an infrared sensor, optical sensor, tactile sensor, a photoplethysmography (PPG) sensor or the like.

Alternatively, the sensor 160 may comprise an ultrasound transducer or probe configured to detect the location of the vagus nerve underlying stimulator 100. The probe may be housed within stimulator 100, or it may be a separate device. The probe may be connected to an ultrasound machine that displays the anatomical structures that lie under the probe. Alternatively, the probe may be coupled to a controller or other device that is configured to provide an indication or alert when the probe has illustrated the carotid sheath.

The control unit 130 controls the impulse generator 110 to generate a signal for each of the device's electrodes (or magnetic coils). The signals are selected to be suitable for amelioration of a particular medical condition when the signals are applied non-invasively to a target nerve or tissue via the electrodes 140. It is noted that nerve stimulating/modulating device 100 may be referred to by its function as a pulse generator. Patent application publications US2005/0075701 and US2005/0075702, both to SHAFER, the disclosure of which is incorporated herein by reference for all purposes as if copied and pasted herein, contain descriptions of pulse generators that may be applicable to this disclosure. By way of example, a pulse generator is also commercially available, such as Agilent 33522A Function/Arbitrary Waveform Generator, Agilent Technologies, Inc., 5301 Stevens Creek Blvd Santa Clara CA 95051.

The control unit 130 may comprise a general purpose computer, comprising one or more CPU, computer memories for the storage of executable computer programs (including the system's operating system) and the storage and retrieval of data, disk storage devices, communication devices (such as serial and USB ports) for accepting external signals from a keyboard, computer mouse, and touchscreen, as well as any externally supplied physiological signals, analog-to-digital converters for digitizing externally supplied analog signals, communication devices for the transmission and receipt of data to and from external devices such as printers and modems that comprise part of the system, hardware for generating the display of information on monitors or display screens that comprise part of the system, and busses to interconnect the above-mentioned components. Thus, the user may operate the system by typing or otherwise providing instructions for the control unit 130 at a device such as a keyboard or touchscreen and view the results on a device such as the system's computer monitor or display screen, or direct the results to a printer, modem, and/or storage disk. Control of the system may be based upon feedback measured from externally supplied physiological or environmental signals. Alternatively, the control unit 130 may have a compact and simple structure, for example, wherein the user may operate the system using only an on/off switch and energy control wheel or knob, or their touchscreen equivalent. In a section below, an embodiment is also described wherein the stimulator housing has a simple structure, but other components of the control unit 130 are distributed into other devices.

Parameters for the nerve or tissue stimulation include energy level, frequency and train duration (or pulse number). The stimulation characteristics of each pulse, such as depth of penetration, strength and selectivity, depend on the rise time and peak electrical energy transferred to the electrodes, as well as the spatial distribution of the electric field that is produced by the electrodes. The rise time and peak energy are governed by the electrical characteristics of the stimulator and electrodes, as well as by the anatomy of the region of current flow within the user. In some embodiments, pulse parameters are set in such a way as to account for the detailed anatomy surrounding the nerve that is being stimulated [Bartosz SAWICKI, Robert Szmurło, Przemysław Płonecki, Jacek Starzyński, Stanisław Wincenciak, Andrzej Rysz. Mathematical Modelling of Vagus Nerve Stimulation. pp. 92-97 in: Krawczyk, A. Electromagnetic Field, Health and Environment: Proceedings of EHE'07. Amsterdam, IOS Press, 2008, the disclosure of which is incorporated herein by reference for all purposes as if copied and pasted herein]. Pulses may be monophasic, biphasic or polyphasic. In some embodiments, some devices include those that are fixed frequency, where each pulse in a train has the same inter-stimulus interval, and those that have modulated frequency, where the intervals between each pulse in a train can be varied.

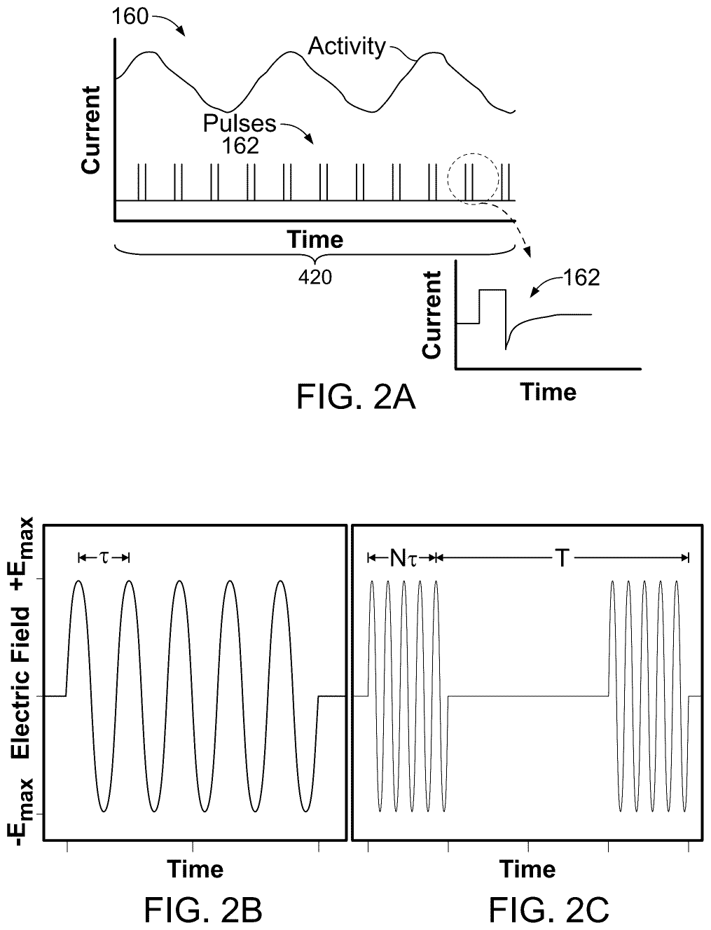

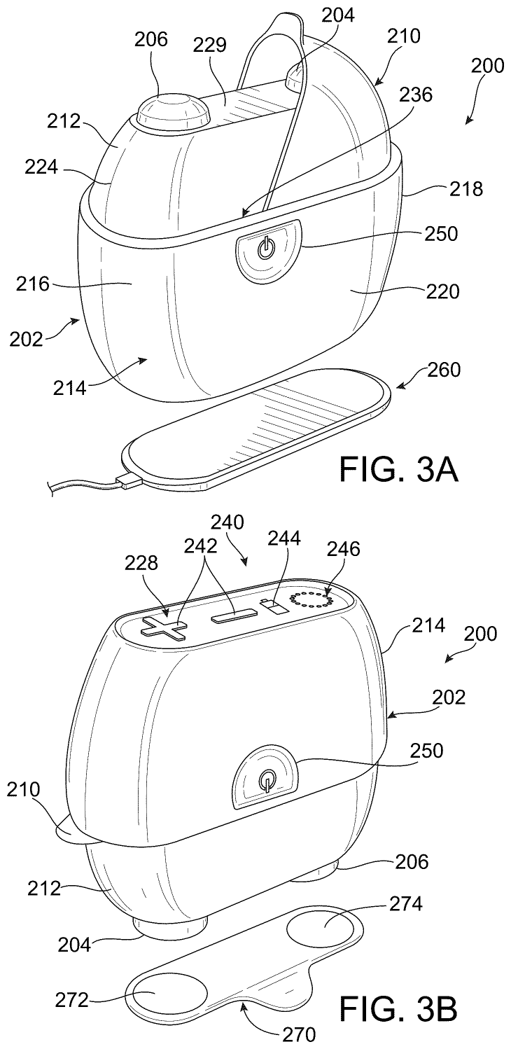

FIG. 2A illustrates an example of an electrical voltage/current profile for a stimulating, blocking and/or modulating impulse applied to a portion or portions of selected nerves in accordance with an embodiment of this disclosure. For some embodiments, the voltage and current refer to those that are non-invasively produced within the user by the electrodes (or magnetic coils). As shown, a suitable electrical voltage/current profile 160 for the blocking and/or modulating impulse 162 to the portion or portions of a nerve may be achieved using pulse generator 110. In some embodiments, the pulse generator 100 may be implemented using an energy source 120 and a control unit 130 having, for instance, a processor, a clock, a memory, etc., to produce a pulse train 164 to the electrodes 140 that deliver the stimulating, blocking and/or modulating impulse 162 to the nerve. Nerve stimulating/modulating device 100 may be externally energized and/or recharged or may have its own energy source 120. The parameters of the modulation signal 160, such as the frequency, amplitude, duty cycle, pulse width, pulse shape, etc., can be programmable, non-programmable, modifiable, locally or remotely updateable, or others. An external communication device may modify the pulse generator programming to improve treatment.

In addition, or as an alternative to some of the devices to implement the modulation unit for producing the electrical voltage/current profile of the stimulating, blocking and/or modulating impulse to the electrodes, the device disclosed in US Patent Application Publication No. US2005/0216062, the disclosure of which is incorporated herein by reference for all purposes as if copied and pasted herein, may be employed. That patent publication discloses a multifunctional electrical stimulation (ES) system adapted to yield output signals for effecting electromagnetic or other forms of electrical stimulation for a broad spectrum of different biological and biomedical applications, which produce an electric field pulse in order to non-invasively stimulate nerves. The system includes an ES signal stage having a selector coupled to a plurality of different signal generators, each producing a signal having a distinct shape, such as a sine wave, a square or a saw-tooth wave, or simple or complex pulse, the parameters of which are adjustable in regard to amplitude, duration, repetition rate and other variables. Examples of the signals that may be generated by such a system are described in a publication by LIBOFF [A. R. LIBOFF. Signal shapes in electromagnetic therapies: a primer. pp. 17-37 in: Bioelectromagnetic Medicine (Paul J. Rosch and Marko S. Markov, eds.). New York: Marcel Dekker (2004), the disclosure of which is incorporated herein by reference for all purposes as if copied and pasted herein]. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated, as well as the outputs of various sensors which sense prevailing conditions prevailing in this substance, whereby the user of the system can manually adjust the signal, or have it automatically adjusted by feedback, to provide an electrical stimulation signal of whatever type the user wishes, who can then observe the effect of this signal on a substance being treated.

The stimulating and/or modulating impulse signal 160 preferably has a frequency, an amplitude, a duty cycle, a pulse width, a pulse shape, etc. selected to influence the therapeutic result, namely, stimulating and/or modulating some or all of the transmission of the selected nerve. For example, the frequency may be about 1 Hz or greater, such as between about 15 Hz to 100 Hz, preferably between about 15-50 Hz and more preferably between about 15-35 Hz. In some embodiments, the frequency is 25 Hz. The modulation signal may have a pulse width selected to influence the therapeutic result, such as about 1 microseconds to about 1000 microseconds, preferably about 100-400 microseconds and more preferably about 200-400 microseconds. For example, the electric field induced or produced by the device within tissue in the vicinity of a nerve may be about 5 to 600 V/m, preferably less than 100 V/m, and even more preferably less than 30 V/m. The gradient of the electric field may be greater than 2 V/m/mm. More generally, the stimulation device produces an electric field in the vicinity of the nerve that is sufficient to cause the nerve to depolarize and reach a threshold for action potential propagation, which is approximately 8 V/m at 1000 Hz. The modulation signal may have a peak voltage amplitude selected to influence the therapeutic result, such as about 0.2 volts or greater, such as about 0.2 volts to about 40 volts, preferably between about 1-20 volts and more preferably between about 2-12 volts.

In an exemplary embodiment, the waveform comprises bursts of sinusoidal pulses, as shown in FIGS. 2B and 2C. As seen there, individual sinusoidal pulses have a period of T, and a burst consists of N such pulses. This is followed by a period with no signal (the inter-burst period). The pattern of a burst followed by silent inter-burst period repeats itself with a period of T. For example, the sinusoidal period T may be between about 50-1000 microseconds with a frequency of about 1-20 kHz), preferably between about 100-400 microseconds with a frequency of about 2.5-10 kHz, more preferably about 133-400 microseconds with a frequency of about 2.5-7.5 kHz and even more preferably about 200 microseconds with a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2E to make the bursts discernable). When these exemplary values are used for T and T, the waveform contains significant Fourier components at higher frequencies ( 1/200 microseconds=5000/sec), as compared with those contained in transcutaneous nerve stimulation waveforms, as currently practiced.

The above waveform is essentially a 1-20 KHz signal that includes bursts of pulses with each burst having a frequency of about 5-100 Hz and each pulse having a frequency of about 1-20 KHz. Another way of thinking about the waveform is that it is a 1-20 KHz waveform that repeats itself at a frequency of about 5-100 Hz.

Invasive nerve stimulation typically uses square wave pulse signals. However, Applicant found that square waveforms are not ideal for non-invasive stimulation, as they produce excessive pain, but still can be used. Prepulses and similar waveform modifications have been suggested as methods to improve selectivity of nerve stimulation waveforms, but Applicant also did not find them ideal, although they still can be used [Aleksandra VUCKOVIC, Marco Tosato and Johannes J Struijk. A comparative study of three techniques for diameter selective fiber activation in the vagal nerve: anodal block, depolarizing prepulses and slowly rising pulses. J. Neural Eng. 5 (2008): 275-286, the disclosure of which is incorporated herein by reference for all purposes as if copied and pasted herein; Aleksandra VUCKOVIC, Nico J. M. Rijkhoff, and Johannes J. Struijk. Different Pulse Shapes to Obtain Small Fiber Selective Activation by Anodal Blocking—A Simulation Study. IEEE Transactions on Biomedical Engineering 51 (5,2004): 698-706, the disclosure of which is incorporated herein by reference for all purposes as if copied and pasted herein; Kristian HENNINGS. Selective Electrical Stimulation of Peripheral Nerve Fibers: Accommodation Based Methods. Ph.D. Thesis, Center for Sensory-Motor Interaction, Aalborg University, Aalborg, Denmark, 2004, the disclosure of which is incorporated herein by reference for all purposes as if copied and pasted herein].

In some embodiments, the use of feedback to generate the modulation signal 160 may result in a signal that is not periodic, particularly if the feedback is produced from sensors that measure naturally occurring, time-varying aperiodic physiological signals from the user. In fact, the absence of significant fluctuation in naturally occurring physiological signals from a user is ordinarily considered to be an indication that the user is in ill health. This is because a pathological control system that regulates the user's physiological variables may have become trapped around only one of two or more possible steady states and is therefore unable to respond normally to external and internal stresses. Accordingly, even if feedback is not used to generate the modulation signal 160, it may be useful to artificially modulate the signal in an aperiodic fashion, in such a way as to simulate fluctuations that would occur naturally in a healthy individual. Thus, the noisy modulation of the stimulation signal may cause a pathological physiological control system to be reset or undergo a non-linear phase transition, through a mechanism known as stochastic resonance [B. SUKI, A. Alencar, M. K. Sujeer, K. R. Lutchen, J. J. Collins, J. S. Andrade, E. P. Ingenito, S. Zapperi, H. E. Stanley, Life-support system benefits from noise, Nature 393 (1998) 127-128, the disclosure of which is incorporated herein by reference for all purposes as if copied and pasted herein; W Alan C MUTCH, M Ruth Graham, Linda G Girling and John F Brewster. Fractal ventilation enhances respiratory sinus arrhythmia. Respiratory Research 2005, 6:41, pp. 1-9, the disclosure of which is incorporated herein by reference for all purposes as if copied and pasted herein].

In some embodiments, the modulation signal 160, with or without feedback, will stimulate the selected nerve fibers in such a way that one or more of the stimulation parameters (e.g., energy, frequency, and others mentioned herein) are varied by sampling a statistical distribution having a mean corresponding to a selected, or to a most recent running-averaged value of the parameter, and then setting the value of the parameter to the randomly sampled value. The sampled statistical distributions will comprise Gaussian and 1/f, obtained from recorded naturally occurring random time series or by calculated formula. Parameter values will be so changed periodically, or at time intervals that are themselves selected randomly by sampling another statistical distribution, having a selected mean and coefficient of variation, where the sampled distributions comprise Gaussian and exponential, obtained from recorded naturally occurring random time series or by calculated formula.

In some embodiments, some devices, as disclosed herein, are provided in a “pacemaker” type form, in which electrical impulses 162 are generated to a selected region of the nerve by a stimulator device on an intermittent basis, to create in the user a lower reactivity of the nerve.

### Embodiments of the Stimulators

The electrodes of the some of the devices, as disclosed herein, are applied to the surface of the neck, or to some other surface of the body, and are used to deliver electrical energy non-invasively to a nerve. Embodiments may differ with regard to the number of electrodes that are used, the distance between electrodes, and whether disk, ring or other shapes of electrodes are used. In some embodiments, one selects the electrode configuration for individual users, in such a way as to optimally focus electric fields and currents onto the selected nerve, without generating excessive currents on the surface of the skin.

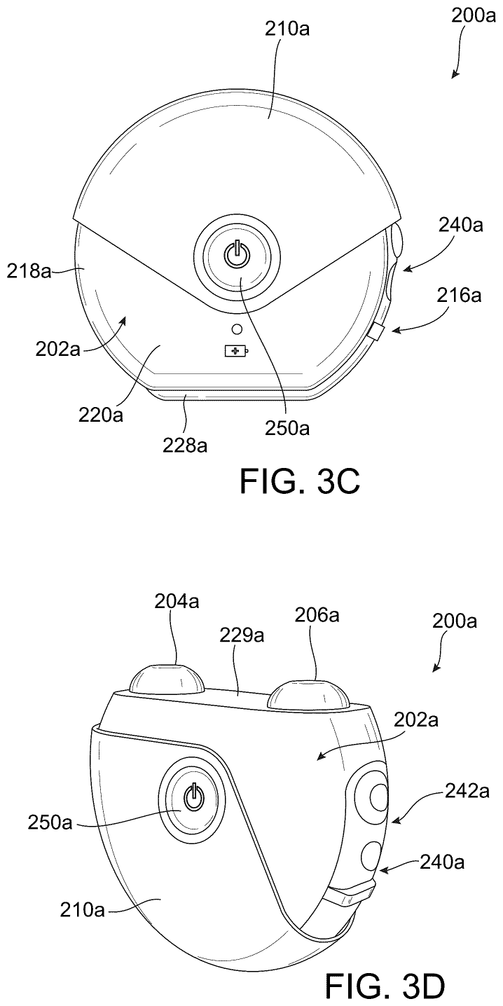

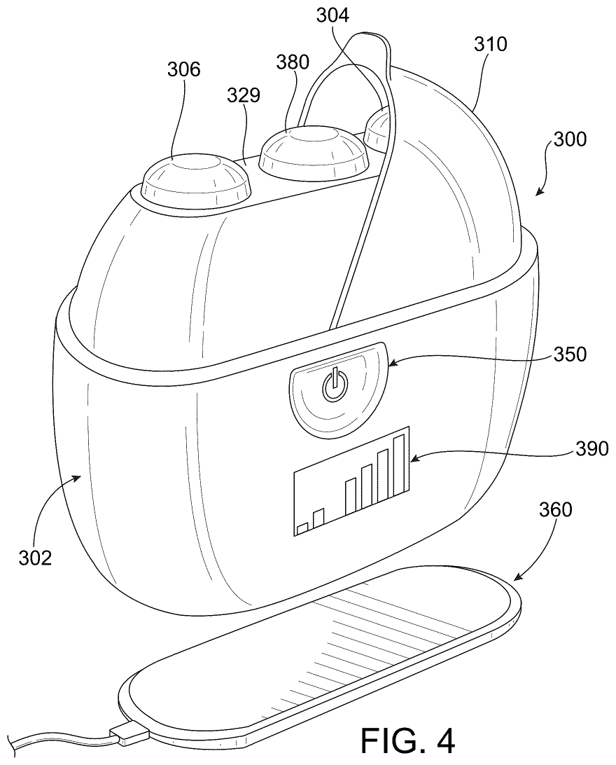

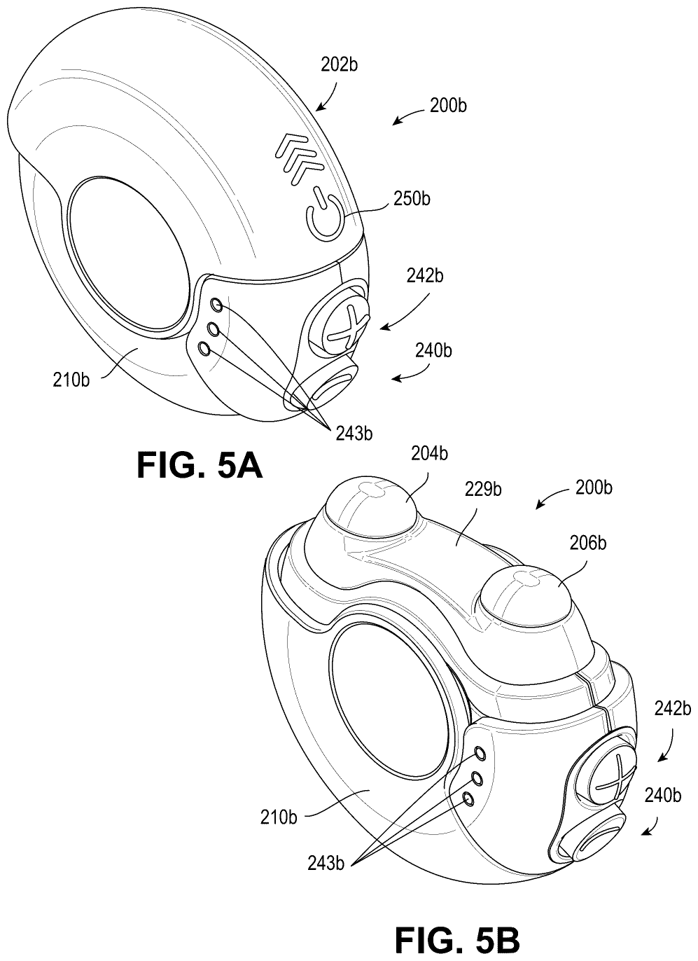

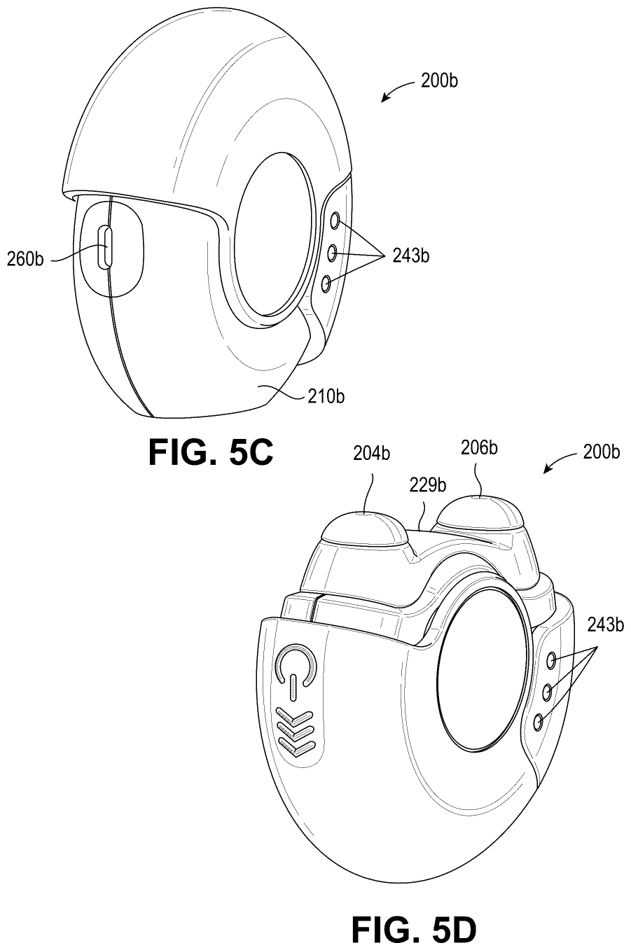

Referring now to FIGS. 3A and 3B, one embodiment of a stimulator 200 comprises a housing 202 and first and second electrodes 204, 206 extending from one surface of housing 202. Electrodes 204, 206 are applied to a surface of the user's body, during which time stimulator 200 may be held in place by straps, frames, collars or the like, or the stimulator 200 may be held against the user's body by hand.

Housing 202 contains the electronic components, signal generator and energy source (not shown) that are used to generate the signals that drive electrical impulses through electrodes 204, 206. However, in other embodiments, the electronic components that generate the signals may be in a separate housing or device, such as a mobile device. Furthermore, other embodiments may contain a single electrode or more than two electrodes.

Housing 202 comprises upper and lower portions 212, 214 and a cover 210 disposed between upper and lower portions 212, 214 for protecting electrodes 204, 206 from the external environment. Cover 210 also ensures that electrodes 204, 206 will not contact a user's tissue when the device is not intended to be used (e.g., in the event that the device is accidently turned ON and electric current is passed through electrodes when not in use). In certain embodiments, cover 210 is rotatably coupled to housing 202 such that it can be moved between a first position, wherein the electrodes 204, 206 are exposed for stimulation, and a second position, wherein the electrodes are housed and protected within the cover 210. Cover 210 may comprise any suitable material, such as polyphenylene ether (PPE), plastic, or other polymers.

Lower portion 214 of housing 202 preferably includes curved side surfaces 216, 218 with substantially planar surfaces 220 therebetween to form an overall disc-like shape that is cut off on the upper and lower portions of the disc. Lower portion 214 also includes a substantially planar bottom surface 228 that includes a control panel 240 (discussed below).

Similarly, upper portion 212 of housing preferably comprises curved side surfaces 224 and a substantially planar upper surface 229. Electrodes 204, 206 extend outward from upper surface 229. Upper portion 212 has a smaller width and length as lower portion 214 to form a groove 236 therebetween. Cover 210 rotates within groove 236. Upper and lower portions 212, 214 are preferably coupled to each other within housing 202. Alternatively, they may be molded together and formed as an integral component.

Control panel 240 may include a number of user controls and/or device status indicators. In alternative embodiments, the controls and status indicators are located on a separate device, such as a mobile device, that is wirelessly (e.g. Bluetooth or the like) coupled to stimulator 200.

In a preferred embodiment, control panel 240 includes intensity controls 242 for controlling the level of intensity or amplitude of the electrical impulses generated by stimulation 200. Intensity controls 242 extend outward from lower surface 228 so that the user be tactically identify and control intensity controls 242.

Control panel 240 may further comprise a battery life indicator 244 and/or a dose duration indicator 246. These indicators may include, for example, LEDs or other light sources, to facilitate identification by the user. The dose duration indicator 246 provides an indication of the time remaining on a single dose of electrical stimulation. In certain embodiments, stimulation 200 is configured to automatically cease the generation of the electrical impulse when the duration of the single dose has been completed.

Stimulator 200 may further include a power control 250 for turning ON the device. Power control 250 may also include an LED or other light source for illuminating power control 250 when the device has been turned ON. In one embodiment, power control 250 is located on side surface 200, although it will be understood that power control 250 may be located on control panel 240 or elsewhere on stimulator 200.

In certain embodiments, stimulator 200 further includes a charging pad 260 coupled to a suitable connector for providing power to stimulator 200 and/or recharging the battery within stimulator 200. Charging pad 260 may comprise any suitable charging source, such as an inductive charging source that provides power via inductive transmission through the lower surface 228 of housing 202.

Stimulator 200 may further include a gel pad 270 that includes a conductive gel 272, 274 positioned to contact electrodes 204, 206 when gel pad 270 is positioned over upper surface 229. Gel pad 270 is configured to apply a coating of electrically conductive gel to the surfaces of electrodes 204, 206 to facilitate conduction of the electrical impulses through an outer skin surface of the user.

The housing 202 may comprise plastic, metal, rubber, or other materials. The housing 202 may be rigid, elastic, resilient, or flexible. The housing 202 may be included in, or embodied as, a phone, a tablet, a laptop, a phone/tablet/laptop case, a patch, an adhesive bandage, a strip, an anklet, a belt, a bracelet, a necklace, a garment, a pad, a ring, a mattress, a pillow, a blanket, a robot, a surgical instrument, a stimulator, an infusion device, or others. The housing 202 may be embodied as described in US Patent Application Publication 20140330336 and U.S. Pat. Nos. 8,874,205, 9,174,066, 9,205,258, 9,375,571, and 9,427,581, all of which are incorporated entirely herein by reference for all purposes as if copied and pasted herein.

Electrodes 204, 206 may comprise a substantially solid conducting material (e.g., metal such as stainless steel, platinum, or a platinum-iridium alloy), which is possibly flexible in some embodiments. However, in other embodiments, the electrodes may have many other sizes and shapes, and they may be made of other materials. The electrodes preferably have a dome-shape with a rounded distal surface, although they may have the shape of a screw that is flattened on its tip. Pointing of the tip would make the electrode more of a point source, such that the equations for the electrical potential may have a solution corresponding more closely to a far-field approximation. Rounding of the electrode surface or making the surface with another shape will likewise affect the boundary conditions that determine the electric field.

In other embodiments, electrodes 204, 206 may be housed within housing 200. In these embodiments, housing includes an outer contact surface, such as a fluid permeable material that allows for passage of current through the permeable portions of the material. In these embodiments, a conductive medium (such as a gel) is preferably situated between the electrode(s) and the permeable interface. The conductive medium provides a conductive pathway for electrons to pass through the permeable interface to the outer surface of the interface and to the user's skin.

In other embodiments, the interface is made from a very thin material with a high dielectric constant, such as material used to make capacitors. For example, it may be Mylar having a submicron thickness (preferably in the range about 0.5 to about 1.5 microns) having a dielectric constant of about 3. Because one side of Mylar is slick, and the other side is microscopically rough, two different configurations are contemplated: one in which the slick side is oriented towards the user's skin, and the other in which the rough side is so oriented. Thus, at stimulation Fourier frequencies of several kilohertz or greater, the dielectric interface will capacitively couple the signal through itself, because it will have an impedance comparable to that of the skin. Thus, the dielectric interface will isolate the stimulator's electrode from the tissue, yet allow current to pass. In one embodiment, non-invasive electrical stimulation of a nerve is accomplished essentially substantially capacitively, which reduces the amount of ohmic stimulation, thereby reducing the sensation the user feels on the tissue surface. This would correspond to a situation, for example, in which at least 30%, preferably at least 50%, of the energy stimulating the nerve comes from capacitive coupling through the stimulator interface, rather than from ohmic coupling. In other words, a substantial portion (e.g., 50%) of the voltage drop is across the dielectric interface, while the remaining portion is through the tissue.

In certain embodiments, stimulator 200 includes an electronic filter, such as a low-pass filter that filters out or eliminates high frequency components from the signal to smooth out the signal before it reaches the electrodes 204, 206. The low-pass filter may comprise a digital or analog filter or simply a capacitor placed in series between the signal generator and the electrode/interface. When the signal is generated, energy switching and electrical noise typically add unwanted high frequency spikes back into the signal. In addition, the pulsing of the sinusoidal bursts may induce high frequency components in the signal. By filtering the signal just before it reaches the electrodes, a smoother, cleaner signal is applied to the user, thereby reducing the pain and discomfort felt by the user and allowing a higher amplitude to be applied to the user. This allows a sufficiently strong signal to be applied to reach a deeper nerve, such as the vagus nerve, without causing too much pain and discomfort to the user at the surface of their skin.

Referring now to FIGS. 3C and 3D, another embodiment of a stimulator 200a comprises a housing 202a and first and second electrodes 204a, 206a extending from one surface of housing 202a. As in the previous embodiment, housing 202a contains the electronic components, signal generator and energy source (not shown) that are used to generate the signals that drive electrical impulses through electrodes 204a, 206a.

Stimulator 200a comprises a cover 210a for protecting electrodes 204a, 206a from the external environment. Cover 210 also ensures that electrodes 204a, 206a will not contact a user's tissue when the device is not intended to be used (e.g., in the event that the device is accidently turned ON and electric current is passed through electrodes when not in use). In certain embodiments, cover 210a is rotatably coupled to housing 202a such that it can be moved between a first position (FIG. 3D), wherein the electrodes 204a, 206a are exposed for stimulation, and a second position (FIG. 3C), wherein the electrodes are housed and protected within the cover 210. Cover 210a may comprise any suitable material, such as polyphenylene ether (PPE), plastic, or other polymers.

Housing 202a preferably includes curved side surfaces 216a, 218a with substantially planar surfaces 220a therebetween to form an overall disc-like shape that is cut off on the upper portion of the disc. A control panel 240a may be included on one of the side surfaces 216a. Control panel 240a includes a number of user controls and/or device status indicators. In alternative embodiments, the controls and status indicators are located on a separate device, such as a mobile device, that is wirelessly (e.g. Bluetooth or the like) coupled to stimulator 200a.

In a preferred embodiment, control panel 240a includes intensity controls 242a for controlling the level of intensity or amplitude of the electrical impulses generated by stimulator 200a. Intensity controls 242a extend outward from side surfaces 216a so that the user may tactically identify and control intensity controls 242a.

Control panel 240a may further comprise a battery life indicator and/or a dose duration indicator (not shown). These indicators may include, for example, LEDs or other light sources, to facilitate identification by the user.

Stimulator 200a may further include a power control 250a for turning ON the device. Power control 250a may also include an LED or other light source for illuminating power control 250a when the device has been turned ON. In one embodiment, power control 250a is located on planar surfaces 220a, although it will be understood that power control 250a may be located on control panel 240a or elsewhere on stimulator 200a.

In certain embodiments, stimulator 200a further includes a charging pad (not shown) coupled to a suitable connector for providing power to stimulator 200a and/or recharging the battery within stimulator 200a. The charging pad may comprise any suitable charging source, such as an inductive charging source that provides power via inductive transmission through the lower surface 228a of housing 202a. Stimulator 200 may further include a gel pad (not shown) that includes a conductive gel positioned to contact electrodes 204a, 206a when gel pad is positioned over upper surface 229a.

Referring now to FIG. 4, another embodiment of a stimulator 300 has a similar construction as stimulator 200 described above. In addition, stimulator 300 includes a sensor 380 extending from upper surface 329 of housing 302. Sensor 380 is preferably located between electrodes 304, 306, although it will be recognized that sensor 380 may be positioned in other locations on housing 302. For example, sensor 380 may be positioned on one of the side surfaces of housing 202, on the bottom surface 328 of housing, or electrodes 304, 306 may be positioned closer together such that sensor 380 is positioned on either side of electrodes 304, 306.

In certain embodiments, sensor 380 comprises a heart pulse sensor that detects the heart pulse of the user when the sensor 380 is placed in contact with, or near, the outer skin surface of the user. As discussed above, the heart pulse sensor detects that the sensor is close to, or adjacent, a source of heart pulse, such as the carotid artery in the user's neck or the radial artery in the wrist. The heart pulse sensor may be any suitable sensor known in the art, for detecting the heart pulse of a user, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 380 is located adjacent to, or near, the user's heart pulse.

Sensor 380 is configured to generate an output that indicates the proximity of a heart pulse in the user. The output may be generated and transmitted via wire, wirelessly, or waveguide, to a control unit within stimulator 300, a mobile device, processor, server, or other logic or computing device. This output provides an indication that electrodes 304, 306 are positioned optimally to modulate the target nerve, e.g., the vagus nerve.

Stimulator 300 further includes a position indicator 390 coupled to sensor 380, the control until within stimulator 300, or a separate device, and configured to provide indication of the position of the stimulator relative to the heart pulse within the user. As discussed above, position indicator is configured to generate an alert when sensor 380 has detected the target nerve. The alert may be, for example, a visual, tactile or audial alert, that provides the user with an indication that the sensor 380 has detected the target location.

In certain embodiments, position indicator 380 is configured to transmit an alert that is associated with the magnitude of the heart pulse. In other embodiments, position indicator 390 is further be configured to provide a second alert when the magnitude of the heart pulse reaches a threshold level associated with optimal positioning of the sensor 380 and/or electrodes 304, 306.

Stimulators 200, 200a and 300 may include additional sensors, such as, for example, biosensors, feedback sensors, chemical sensors, optical sensors, acoustic sensors, vibration sensors, motion sensors, fluid sensors, radiation sensors, temperature sensors, motion sensors, proximity sensors, fluid sensors, or others. The sensors may generate an output, such as one or more outputs, which are communicated, via wire, wirelessly or waveguide, to the stimulator 200, a mobile device, processor, server, or other logic or computing device. The output may be used as an input to one or more of the foregoing devices to forecast or avert an imminent onset or predicted upcoming onset of a symptom, episode, condition or disease. For example, as disclosed in U.S. Patent App. Pub. No. 2017/0120052, which is incorporated herein by reference in its entirety for at least these purposes as if copied and pasted herein, as disclosed herein, and for all purposes as if copied and pasted herein, such as all structures, all functions, and all methods of manufacture and use, as disclosed therein.

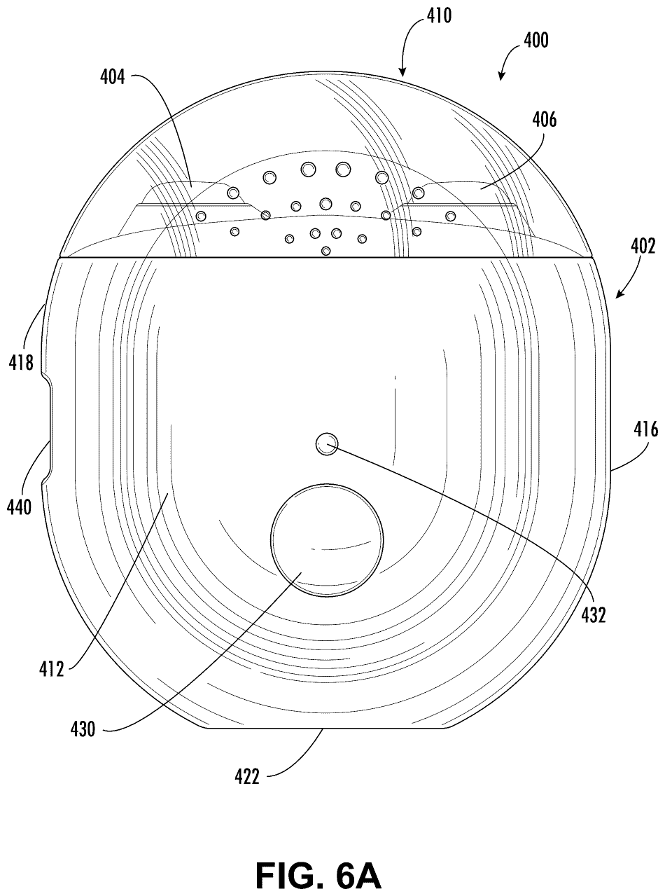

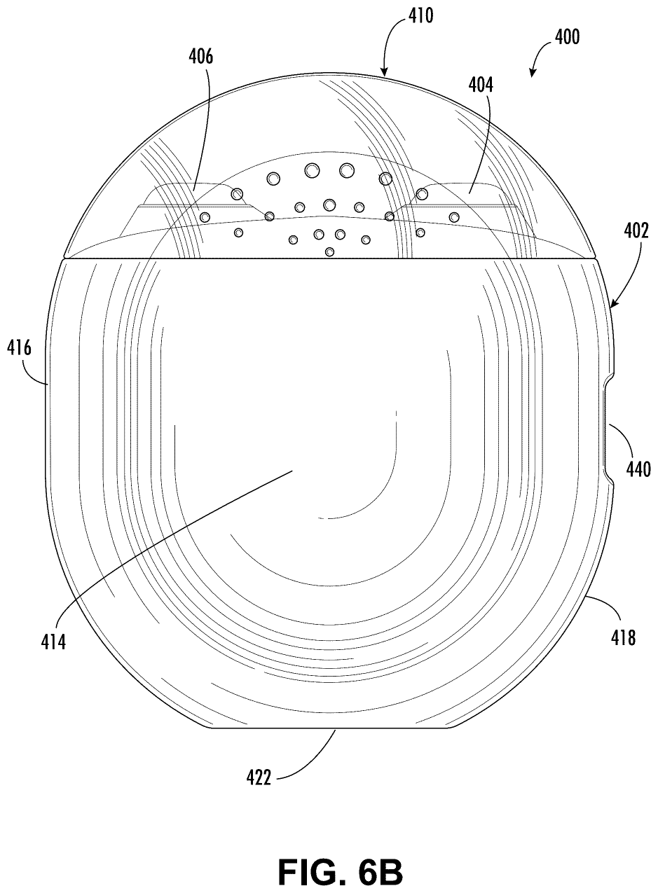

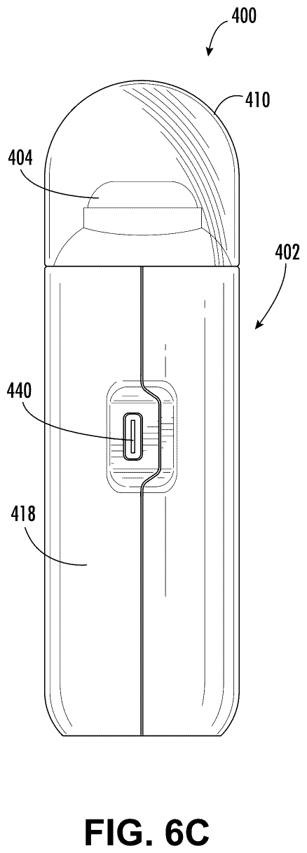

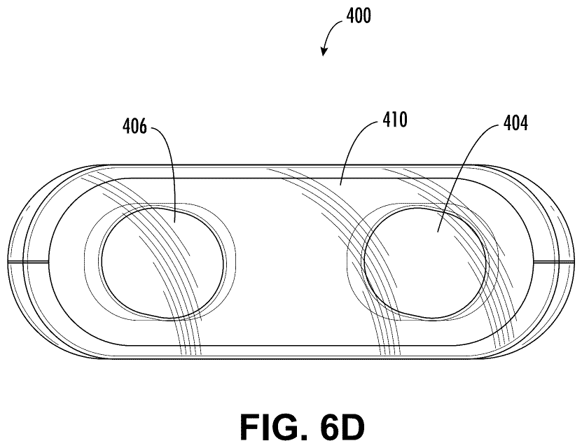

Referring now to FIGS. 6A-6D, another embodiment of a stimulator 400 comprises a housing 402 and first and second electrodes 404, 406 extending from one surface of housing 402. As in the previous embodiment, housing 402 contains the electronic components, signal generator and energy source (not shown) that are used to generate the signals that drive electrical impulses through electrodes 404, 406.

Stimulator 400 comprises a transparent cover 410 for protecting electrodes 404, 406 from the external environment. Cover 410 also ensures that electrodes 404, 406 will not contact a user's tissue when the device is not intended to be used (e.g., in the event that the device is accidently turned ON and electric current is passed through electrodes when not in use). In certain embodiments, cover 410 is rotatably coupled to housing 402 as in previous embodiments. In other embodiments, cover 410 is removably coupled to housing 402 so that it can be removed by the user prior to application of the stimulation signal. Cover 410 may comprise any suitable material, such as polyphenylene ether (PPE), plastic, or other polymers.

Housing 402 preferably includes substantially planar front and rear surfaces 412, 414 and side surfaces 416, 418. A control panel may be included on one of the surfaces of housing 402. The control panel includes a number of user controls for adjusting the parameters of the stimulation, such as the intensity of the electrical impulse. In a preferred embodiment, the control panel includes a power button 430 located on front surface 412. For example, housing 402 may include intensity controls for controlling the level of intensity or amplitude of the electrical impulses generated by stimulator 400. The intensity controls extend outward from side surfaces 416, 418, or from front or rear surfaces 412, 414 so that the user may tactically identify and control intensity controls 240b, 242b.

The control panel may also include a number of status indicators 432 for providing status information to the user relative to the device. These indicators may include, for example, LEDs or other light sources, to facilitate identification by the user. In one embodiment, status indicators 434 may include LED lights that indicate battery life (e.g., with different colors or with flashing to indicate low battery life), the time remaining in a single dose (e.g., changing colors or flashing when the time remaining reaches a certain level), the intensity level (i.e., with changing colors or flashing) or other parameters of the electrical signal. In alternative embodiments, the controls and status indicators are located on a separate device, such as a mobile device, that is wirelessly (e.g. Bluetooth or the like) coupled to stimulator 400.

Stimulator 200b further includes an electrical connection 440, such as a USB or the like, for providing power to stimulator 400 and/or recharging the battery within stimulator 400. Electrical connection 440 is preferably positioned on side surface 418

In certain embodiments, stimulator 400 further includes a charging pad (not shown) coupled to a suitable connector for providing power to stimulator 400 and/or recharging the battery within stimulator 400. The charging pad may comprise any suitable charging source, such as an inductive charging source that provides power via inductive transmission through the lower surface 422 of housing 402. Stimulator 400 may further include a gel pad (not shown) that includes a conductive gel positioned to contact electrodes 404, 406 when gel pad is positioned over housing 402.

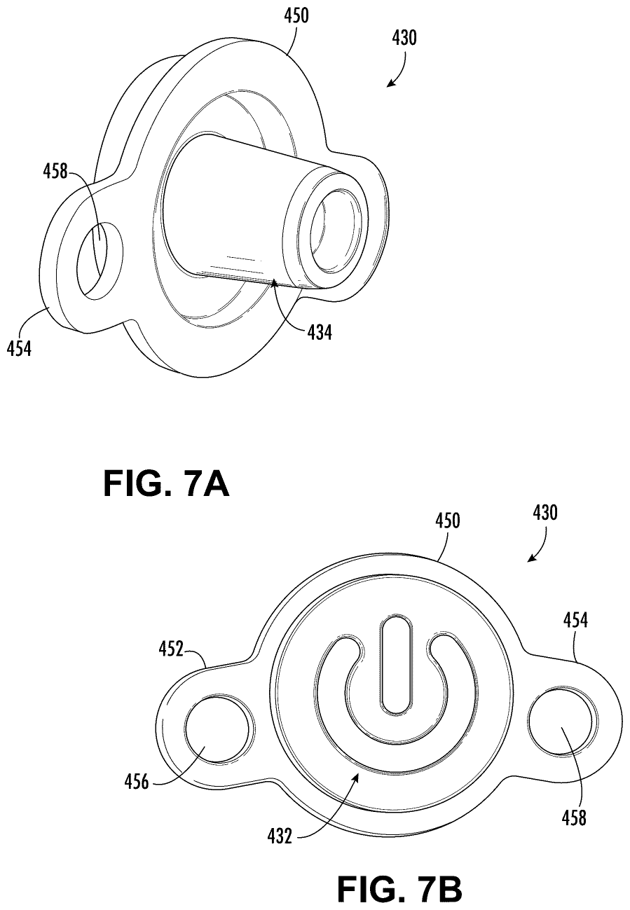

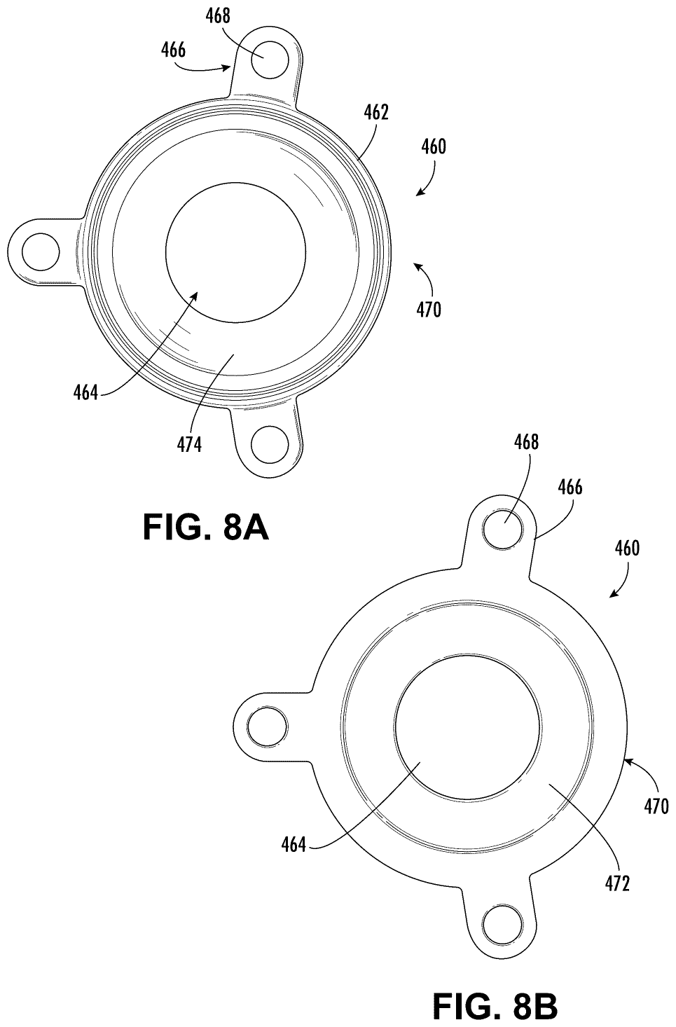

Referring now to FIGS. 7A, 7B, 8A and 8B, a preferred embodiment of power control button 430 will now be described. As shown, control button 430 comprises a main body 450 having a cylindrical projection 434 extending into housing 402, and a pair of arms 452, 454 extending from main body 450. Arms 452, 454 each include an opening 456, 458 extending therethrough. Main body 450 further includes a user interface 432 designed to face the exterior of housing 402 and providing a touch surface that allows the user to press interface 432 to turn the stimulator 400 ON and OFF.

Referring now to FIGS. 8A and 8B, power control button 430 further includes a support member 460 disposed between button 430 and housing 402. Support member 460 couples button 430 to housing 402 and comprises a substantially circular main body 470 with a central channel 464 for receiving projection 434 of button 430. Main body 470 further includes a front surface 462 facing button 430 and having a central portion 474 that tapers inwardly towards channel 464. Main body 470 includes a back recessed surface 472 facing housing 402. Support member 460 further includes one or more support arms 466 extending from main body 470, each having a central opening 468.

Stimulation may be performed on the left or right vagus nerve or on both of them simultaneously and alternately. The position and angular orientation of the device are adjusted about that location until the user perceives stimulation when current is passed through the stimulator electrodes. The applied current is increased gradually, first to a level wherein the user feels sensation from the stimulation. The energy is then increased, but is set to a level that is less than one at which the user first indicates any discomfort. Straps, harnesses, or frames may be used to maintain the stimulator in position. The stimulator signal may have a frequency and other parameters that are selected to produce a therapeutic result in the user, i.e., stimulation parameters for each user are adjusted on an individualized basis. Ordinarily, the amplitude of the stimulation signal is set to the maximum that is comfortable for the user, and then the other stimulation parameters are adjusted.

The stimulation is then performed with a sinusoidal burst waveform like that shown in FIG. 2. As seen there, individual sinusoidal pulses have a period of T, and a burst consists of N such pulses. This is followed by a period with no signal (the inter-burst period). The pattern of a burst followed by silent inter-burst period repeats itself with a period of T. For example, the sinusoidal period T may be between about 50-1000 microseconds and a frequency of about 1-20 kHz, preferably between about 100-400 microseconds and a frequency of about 2.5-10 kHz, more preferably about 133-400 microseconds and a frequency of about 2.5-7.5 kHz and even more preferably about 200 microseconds and a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2C to make the bursts discernable). When these example values are used for T and T, the waveform contains significant Fourier components at higher frequencies ( 1/200 microseconds=5000/sec), as compared with those contained in transcutaneous nerve stimulation waveforms.

### Electronics and Software of the Stimulator

In some embodiments, the signal waveform (FIG. 2) that is to be applied to electrodes of the stimulator is initially generated in a component of an impulse generator that is exterior to, and remote from, a mobile phone housing. The mobile phone preferably includes a software application that can be downloaded (e.g., mobile app store, USB cable, memory stick, Bluetooth connection) into the phone to receive, from the external control component, a wirelessly transmitted waveform, or to receive a waveform that is transmitted by cable, e.g., via a multi-purpose jack. If the waveforms are transmitted in compressed form, they are preferably compressed in a lossless manner, e.g., making use of FLAC (Free Lossless Audio Codec). Alternatively, the downloaded software application may itself be coded to generate a particular waveform that is to be applied to the electrodes and subsequently conveyed to the external interface of the electrode assembly. In some embodiments, the software application is not downloaded from outside the device, but is instead available internally, for example, within read-only-memory that is present within the housing of the stimulator.

In some embodiments, the waveform is first conveyed by the software application to contacts within the phone's speaker output or the earphone jack socket, as though the waveform signal were a generic audio waveform. That pseudo-audio waveform will generally be a stereo waveform, representing signals that are to be applied to the “left” and “right” electrodes. The waveform will then be conveyed to the housing of the stimulator. as follows. The housing of the stimulator may have an attached dangling audio jack that is plugged into the speaker output or the earphone jack socket whenever electrical stimulation is to be performed, or the electrical connection between the contacts of the speaker output or the earphone jack socket and the housing of the stimulator may be hard-wired. In either case, electrical circuits on a printed circuit board located under the belly of the housing of the stimulator may then shape, filter, and/or amplify the pseudo-audio signal that is received via the speaker output or earphone jack socket. An energy amplifier within the housing of the stimulator may then drive the signal onto the electrodes, in a fashion that is analogous to the use of an audio energy amplifier to drive loudspeakers. Alternatively, the signal processing and amplification may be implemented in a separate device that can be plugged into sockets on the phone and/or housing of the stimulator, to couple the software application and the electrodes.

In addition to passing the stimulation waveform from the smartphone to the stimulator housing as described herein, the smartphone may also pass control signals to the stimulator housing. Thus, the stimulation waveform may generally be regarded as a type of analog, pseudo-audio signal, but if the signal contains a signature series of pulses signifying that a digital control signal is about to be sent, logic circuitry in the stimulator housing may then be set to decode the series of digital pulses that follows the signature series of pulses, analogous to the operation of a modem.

Many of the steps that direct the waveform to the electrodes, including steps that may be controlled by the user via the touchscreen, are implemented in the above-mentioned software application. By way of example, the software application may be written for a phone that uses the Android operating system. Such applications are typically developed in the Java programming language using the Android Software Development Kit (SDK), in an integrated development environment (IDE), such as Eclipse [Mike WOLFSON. Android Developer Tools Essentials. Sebastopol, California: O'Reilly Media Inc., 2013; Ronan SCHWARZ, Phil Duston, James Steele, and Nelson To. The Android Developer's Cookbook. Building Applications with the Android SDK, Second Edition. Upper Saddle River, NJ: Addison-Wesley, 2013, the disclosure of which is incorporated herein by reference for all purposes as if copied and pasted herein; Shane CONDER and Lauren Darcey. Android Wireless Application Development, Second Edition. Upper Saddle River, NJ: Addison-Wesley, 2011; Jerome F. DIMARZIO. Android—A Programmer's Guide. New York: McGraw-Hill. 2008. pp. 1-319, the disclosure of which is incorporated herein by reference for all purposes as if copied and pasted herein]. Application programming interfaces (APIs) that are particularly relevant to the audio features of such an Android software application (e.g., MediaPlayer APIs) are described by: Android Open Source Project of the Open Handset Alliance. Media Playback, at web domain developer.android.com with subdomain/guide/topics/media/, Jul. 18, 2014, the disclosure of which is incorporated herein by reference for all purposes as if copied and pasted herein. Those APIs can be relevant to a use of the smartphone camera capabilities, as described below. Additional components of the software application are available from device manufacturers [Samsung Mobile SDK, at web domain developer.samsung.com with subdomain/samsung-mobile-sdk, Jul. 18, 2014, the disclosure of which is incorporated herein by reference for all purposes as if copied and pasted herein].

In some embodiments, the stimulator, the smartphone and/or the wearable electronic device will include a user control, such as a switch or button, that disables/enables the stimulator. Preferably, the switch will automatically disable some, many, most, or all smartphone or the wearable electronic device functions when the stimulator is enabled (and vice versa). This ensures that the medical device functionality of the smartphone or other device is completely segregated from the rest of the phone's functionality. In some embodiments, the switch will be password-controlled such that only the user/owner of the stimulator/phone will be able to enable the stimulator functionality. In one such embodiment, the switch will be controlled by a biometric scan (e.g., fingerprint, optical scan or the like) such that the stimulator functionality can only be used by the user. This ensures that only the user will be able to use the prescribed therapy in the event the phone is lost or stolen.

The stimulator and/or phone can also include software that allows the user to order more therapy doses over the internet (discussed in more detail below in connection with the docking station). The purchase of such therapy doses will require physician authorization through a prescription or the like. To that end, the software can include an authorization code for entry in order for the user to download authorization for more therapies. In some embodiments, without such authorization, the stimulator will be disabled and will not deliver therapy.

In some embodiments, the housing of the stimulator may also be joined to and/or energized by a wireless device that is not a phone (e.g., Wi-Fi enabled device, wearable electronic device, tablet). Alternatively, the stimulator may be coupled to a phone or other Wi-Fi enabled device through a wireless connection for exchanging data at short distances, such as Bluetooth or the like. In this embodiment, the stimulator housing is not attached to the smartphone and, therefore, may comprise a variety of other shapes and sizes that are convenient for the user to carry in his or her purse, wallet or pocket.

In some embodiments, the stimulator housing may be designed as part of a protective or decorative case for the phone that can be attached to the phone, similar to standard phone cases. In one such embodiment, the stimulator/case may also include additional battery life for the phone and may include an electrical connection to the phone's battery to recharge the battery (e.g., part of a Mophie® or the like). This electrical connection may also be used to couple the smartphone to the stimulator.

### Embodiments With Distributed Controllers

In some embodiments, at least some (if not all) portions of the control of the vagus nerve stimulation reside in controller components that are physically separate from the housing of the stimulator. In these embodiment, separate components of the controller and stimulator housing generally communicate with one another wirelessly, although wired or waveguide communication is possible. Suitable components include a remote computer or server, wearable computing devices, such as a smartwatch, Whoop®, Fitbit®, Garmin® or the like, a mobile phone, a mobile processing device (e.g., laptop computers or tablets) and the like. Thus, the use of wireless technology avoids the inconvenience and distance limitations of interconnecting cables.

First, the stimulator may be constructed with the minimum number of components needed to generate the stimulation pulses, with the remaining components placed in parts of the controller that reside outside the stimulator housing, resulting in a lighter and smaller stimulator housing. In fact, the stimulator housing may be made so small that it could be difficult to place, on the stimulator housing's exterior, switches and knobs that are large enough to be operated easily. Instead, the user may generally operate the device using the smartphone touchscreen.

Second, the controller 130 may be given additional functions when free from the limitation of being situated within or near the stimulator housing. For example, one may add to the controller a data logging component that records when and how stimulation has been applied to the user, for purposes of medical recordkeeping and billing. The complete electronic medical record database for the user may be located far from the stimulator (e.g., somewhere on the internet), and the billing system for the stimulation services that are provided may also be elsewhere, so it would be useful to integrate the controller into that recordkeeping and billing system, using a communication system that includes access to the internet or telephone networks.

Third, communication from the databases to the controller would also be useful for purposes of metering electrical stimulation of the user, when the stimulation is self-administered. For example, if the prescription for the user only permits only a specified amount of stimulation energy to be delivered during a single session of vagus nerve stimulation, followed by a wait-time before allowing the next stimulation, the controller can query the database and then permit the stimulation only when the prescribed wait-time has passed. Similarly, the controller can query the billing system to assure that the user's account is in order, and withhold the stimulation if there is a problem with the account.

Fourth, as a corollary of the previous considerations, the controller may be constructed to include a computer program separate from the stimulating device, in which the databases are accessed via cell phone or internet connections.

Fifth, in some applications, it may be desired that the stimulator housing and parts of the controller be physically separate. For example, when the user is a child, one wants to make it impossible for the child to control or adjust the vagus nerve stimulation. The best arrangement in that case is for the stimulator housing to have no touchscreen elements, control switches or adjustment knobs that could be activated by the child. Alternatively, any touchscreen elements, switches and knobs on the stimulator can be disabled, and control of the stimulation then resides only in a remote controller with a child-proof operation, which would be maintained under the control of a parent or healthcare provider.

Sixth, in some applications, the particular control signal that is transmitted to the stimulator by the controller will depend on physiological and environmental signals that are themselves transmitted to and analyzed by the controller. In such applications, many of the physiological and environmental signals may already be transmitted wirelessly, in which case it is most convenient to design an external part of the controller as the hub of all such wireless activity, including any wireless signals that are sent to and from the stimulator housing.

With these considerations in mind, an embodiment of can include a mobile device that may send/receive data to/from the stimulator, and may send/receive data to/from databases and other components of the system, including those that are accessible via the internet (or another network such as local area, wide area, satellite, cellular). Typically, the mobile device will be a laptop computer attached to additional components needed for it to accomplish its function. Thus, prior to any particular stimulation session, the mobile device may load into the stimulator parameters of the session, including waveform parameters, or the actual waveform.

In some embodiments, the mobile device is also used to limit the amount of stimulation energy that may be consumed by the user during the session, by charging the stimulator's rechargable battery with only a specified amount of releasable electrical energy, which is different than setting a parameter to restrict the duration of a stimulation session. Thus, the mobile device may comprise a energy supply that may be connected to the stimulator's rechargable battery, and the mobile device meters the recharge. As a practical matter, the stimulator may therefore use two batteries, one for applying stimulation energy to the electrodes (the charge of which may be limited by the mobile device) and the other for performing other functions. Methods for evaluating a battery's charge or releasable energy can be as disclosed in patent U.S. Pat. No. 7,751,891, entitled Energy supply monitoring for an implantable device, to ARMSTRONG et al, the disclosure of which is incorporated herein by reference for all purposes as if copied and pasted herein. Alternatively, some control components within the stimulator housing may monitor the amount of electrode stimulation energy that has been consumed during a stimulation session and stop the stimulation session when a limit has been reached, irrespective of the time when the limit has been reached.

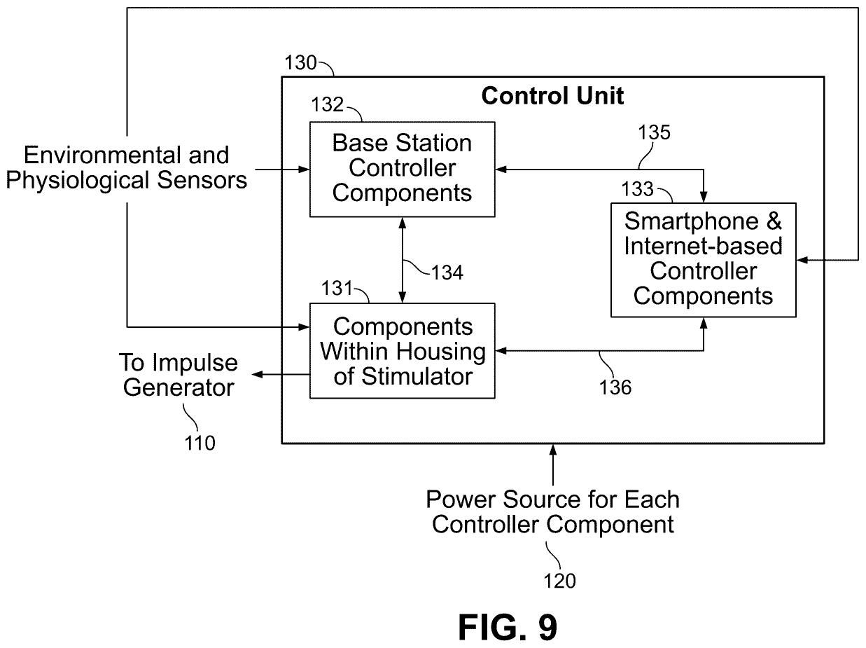

The communication connections between different components of the stimulator's controller are shown in FIG. 9, which is an expanded representation of the control unit 130 in FIG. 1. Connection between the mobile device controller components 132 and components within the stimulator housing 131 is denoted in FIG. 8 as 134. Connection between the mobile device controller components 132 and internet-based (or network based) or smartphone and/or wearable electronic components 133 is denoted as 135. Connection between the components within the stimulator housing 331 and internet-based or smartphone components 133 is denoted as 136. For example, control connections between the smartphone and stimulator housing via the audio jack socket would fall under this category, as would any wireless communication directly between the stimulator housing itself and a device situated on the internet. In principle, the connections 134, 135 and 136 in FIG. 8 may be either wired or wireless or waveguide-based. Different embodiments may lack one or more of the connections.

Although infrared or ultrasound wireless control might be used to communicate between components of the controller, they are not preferred because of line-of-sight limitations. Instead, the communication between devices preferably makes use of radio communication within unlicensed ISM frequency bands (260-470 MHZ, 902-928 MHz, 2400-2.4835 GHZ). Components of the radio frequency system in devices in 331, 332, and 333 typically comprise a system-on-chip transciever with an integrated microcontroller; a crystal; associated balun & matching circuitry, and an antenna [Dag GRINI. RF Basics, RF for Non-RF Engineers. Texas Instruments, Post Office Box 655303, Dallas, Texas 75265, 2006, the disclosure of which is incorporated herein by reference for all purposes as if copied and pasted herein].

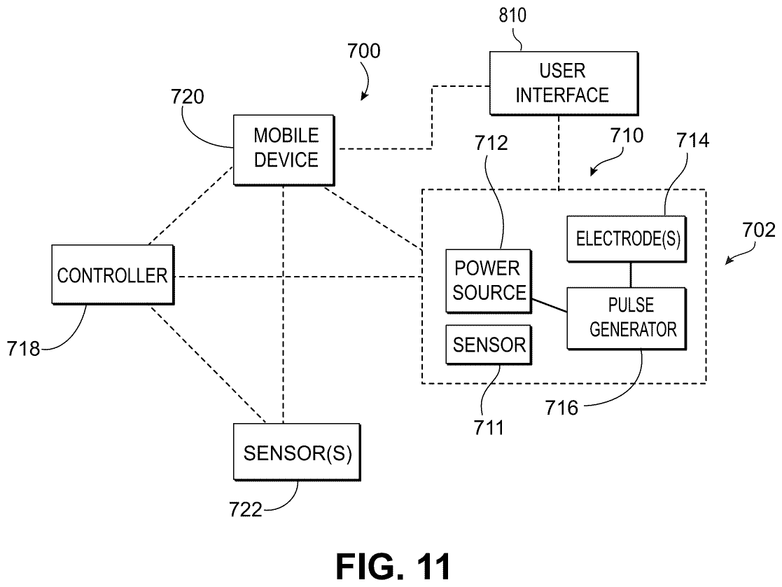

Transceivers based on 2.4 GHz offer high data rates (greater than 1 Mbps) and a smaller antenna than those operating at lower frequencies, which makes them suitable for with short-range devices. Furthermore, a 2.4 GHz wireless standard (e.g., Bluetooth, Wi-Fi, and ZigBee) may be used as the protocol for transmission between devices. Although the ZigBee wireless standard operates at 2.4 GHZ in most jurisdictions worldwide, it also operates in the ISM frequencies 868 MHz in Europe, and 915 MHz in the USA and Australia. Data transmission rates vary from 20 to 250 kilobits/second with that standard. Because many commercially available health-related sensors may operate using ZigBee, its use may be recommended for applications in which the controller uses feedback and feedforward methods to adjust the user's vagus nerve stimulation based on the sensors' values, as described below in connection with FIG. 11 [ZigBee Wireless Sensor Applications for Health, Wellness and Fitness. ZigBee Alliance 2400 Camino Ramon Suite 375 San Ramon, CA 94583].

A 2.4 GHz radio has higher energy consumption than radios operating at lower frequencies, due to reduced circuit efficiencies. Furthermore, the 2.4 GHz spectrum is crowded and subject to significant interference from microwave ovens, cordless phones, 802.11b/g wireless local area networks, Bluetooth devices, etc. Sub-GHz radios enable lower energy consumption and can operate for years on a single battery. These factors, combined with lower system cost, make sub-GHz transceivers ideal for low data rate applications that need maximum range and multi-year operating life.

The antenna length needed for operating at different frequencies is 17.3 cm at 433 MHz, 8.2 cm at 915 MHz, and 3 cm at 2.4 GHz. Therefore, unless the antenna is included in a neck collar that supports the device shown in FIG. 3, the antenna length may be a disadvantage for 433 MHz transmission. The 2.4 GHz band has the advantage of enabling one device to serve in all major markets worldwide since the 2.4 GHz band is a global spectrum standard. However, 433 MHz is a viable alternative to 2.4 GHz for most of the world, and designs based on 868 and 915 MHz radios can serve the US and European markets with a single product.

Range is determined by the sensitivity of the transceiver and its output energy. A primary factor affecting radio sensitivity is the data rate. Higher data rates reduce sensitivity, leading to a need for higher output energy to achieve sufficient range. For many applications that require only a low data rate, the preferred rate is 40 Kbps where the transceiver can still use a standard off-the-shelf 20 parts per million crystal.

A signal waveform that might be transmitted wirelessly to the stimulator housing was shown in FIGS. 2B and 2C. As seen there, individual sinusoidal pulses have a period of tau, and a burst consists of N such pulses. This is followed by a period with no signal (the inter-burst period). The pattern of a burst followed by silent inter-burst period repeats itself with a period of T. For example, the sinusoidal period tau may be 200 microseconds; the number of pulses per burst may be N=5; and the whole pattern of burst followed by silent inter-burst period may have a period of T=40000 microseconds, which is comparable to 25 Hz stimulation (a much smaller value of T is shown in FIG. 2C to make the bursts discernable). When these exemplary values are used for T and tau, the waveform contains significant Fourier components at higher frequencies ( 1/200 microseconds=5000/sec). Such a signal may be easily transmitted using 40 Kbps radio transmission. Compression of the signal is also possible, by transmitting only the signal parameters tau, N, T, Emax, etc., but in that case the stimulator housing's control electronics would then have to construct the waveform from the transmitted parameters, which would add to the complexity of components of the stimulator housing.

However, because it is contemplated that sensors attached to the stimulator housing may also be transmitting information, the data transfer requirements may be substantially greater than what is required only to transmit the signal shown in FIG. 2. Therefore, the devices and methods disclosed herein may make use of any frequency band, not limited to the ISM frequency bands, as well as techniques known in the art to suppress or avoid noise and interferences in radio transmission, such as frequency hopping and direct sequence spread spectrum.

When a user is using the stimulation device to perform self-stimulation therapy, e.g., at home or at a workplace, he or she will follow the steps that are now described. It is assumed that the optimal stimulation position has already been marked on the user's neck, as described above and that a reference image of the fluorescent spots has already been acquired. The previous stimulation session will ordinarily have discharged the rechargeable batteries of the stimulator housing, and between sessions, the mobile device will have been used to recharge the stimulator at most only up to a minimum level. If the stimulator's batteries had charge remaining from the previous stimulation session, the mobile device will discharge the stimulator to a minimum level that will not support stimulation of the user.

The user can initiate the stimulation session using a wearable computing device, such as a smartwatch, Whoop® or the like, a Fitbit®, Garmin® or the like, a mobile phone, a mobile processing device (e.g., laptop computer) and the like, by invoking a computer program (on the laptop computer or through an app on the mobile phone) that is designed to initiate use of the stimulator. The programs in the smartphone and mobile device may initiate and interact with one another wirelessly, so in what follows, reference to the program (app) in the smartphone may also apply to the program in the mobile device, because both may be operating in tandem. For security reasons, the program would begin with the request for a user name and a password, and that user's demographic information and any data from previous stimulator experiences would already be associated with it in the login account. The smartphone may also be used to authenticate the user using a fingerprint or voice recognition app, or other reliable authentication methods. If the user's physician has not authorized further treatments, the mobile device will not charge the stimulator's batteries, and instead, the computer program will call or otherwise communicate with the physician's computer requesting authorization. After authorization by the physician is received, the computer program (on the laptop computer or through an app on the mobile phone) may also query a database that is ordinarily located somewhere on the internet to verify that the user's account is in order. If it is not in order, the program may then request prepayment for one or more stimulation sessions, which would be paid by the user using a credit card, debit card, PayPal, cryptocurrency, bitcoin, or the like. The computer program will also query its internal database or that of the mobile device to determine that sufficient time has elapsed between when the stimulator was last used and the present time, to verify that any required wait-time has elapsed.

Having received authorization to perform a nerve stimulation session, the user interface computer program will then ask the user questions that are relevant to the selection of parameters that the mobile device will use to make the stimulator ready for the stimulation session. The questions that the computer program asks are dependent on the condition for which the user is being treated, which for present purposes is considered to be treatment for an autoimmune disease or disorder. The questions may be things like (1) is this an acute or prophylactic treatment? (2) if acute, then how severe is your pain and in what locations, how long have you had it, (3) has anything unusual or noteworthy occurred since the last stimulation? etc.

Having received such preliminary information from the user, the computer programs will perform instrument diagnostic tests and make the stimulator ready for the stimulation session. In general, the algorithm for setting the stimulator parameters will have been decided by the physician and will include the extent to which the stimulator batteries should be charged, which the vagus nerve should be stimulated (right or left), and the time that the user should wait after the stimulation session is ended until initiation of a subsequent stimulation session. The computer will query the physician's computer to ascertain whether there have been any updates to the algorithm, and if not, will use the existing algorithm. The user will also be advised of the stimulation session parameter values by the interface computer program, so as to know what to expect.

Once the mobile device has been used to charge the stimulator's batteries to the requisite charge, the computer program (or smartphone app) will indicate to the user that the stimulator is ready for use. At that point, the user would clean the electrode surfaces, and make any other preliminary adjustments to the hardware. The stimulation parameters for the session will be displayed, and any options that the user is allowed to select may be made. Once the user is ready to begin, he or she will press a “start” button on the touchscreen and may begin the vagus nerve stimulation.

Multiple methods may be used to test whether the user is properly attempting to stimulate the vagus nerve (or another nerve or organ or muscle or bone) on the intended side of the neck (or another portion of a human body). For example, accelerometers and gyroscopes within the smartphone may be used to determine the position and orientation of the smartphone's touch screen relative to the user's expected view of the screen, and a decision by the stimulator's computer program as to which hand is being used to hold the stimulator may be made by measuring capacitance on the outside of the stimulator body, which may distinguish fingers wrapped around the device versus the ball of a thumb [Raphael WIMMER and Sebastian Boring. HandSense: discriminating different ways of grasping and holding a tangible user interface. Proceedings of the 3rd International Conference on Tangible and Embedded Interaction, pp. 359-362. ACM New York, NY, 2009, the disclosure of which is incorporated herein by reference for all purposes as if copied and pasted herein]. Pressing of the electrodes against the skin will result in a resistance drop across the electrodes, which can initiate operation of the rear camera. A fluorescent image should appear on the smartphone screen only if the device is applied to the side of the neck in the vicinity of the fluorescent spots that had been applied as a tattoo earlier. If the totality of these data indicates to the computer program that the user is attempting to stimulate the wrong vagus nerve or that the device is being held improperly, the stimulation will be withheld, and the stimulator may then communicate with the user via the interface computer program (in the mobile phone or laptop computer) to alert the user of that fact. T

Before logging off of the interface computer program, the user may also review database records and summaries about all previous treatment sessions, so as to make his or her own judgment about treatment progress. If the stimulation was part of a prophylactic treatment regimen that was prescribed by the user's physician, the user interface computer program will remind the user about the schedule for the upcoming self-treatment sessions and allow for a rescheduling if necessary.

For some users, the stimulation may be performed for as little as 60 seconds, but it may also be for up to 30 minutes or longer. The treatment is generally performed once or twice daily or several times a week, for 12 weeks or longer before a decision is made as to whether to continue the treatment. For users experiencing intermittent symptoms, the treatment may be performed only when the user is symptomatic. However, it is understood that parameters of the stimulation protocol may be varied in response to heterogeneity in the pathophysiology of users. Different stimulation parameters may also be used as the course of the user's condition changes.

### Applications of Stimulators to the User

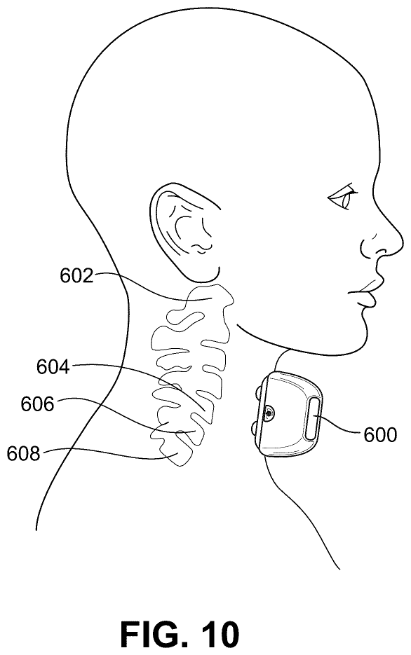

Selected nerve fibers are stimulated in different embodiments of methods that make use of the disclosed electrical stimulation devices, including stimulation of the vagus nerve at a location in the user's neck. FIG. 10 illustrates use of a stimulator 600 to stimulate the vagus nerve at that location in the neck, in which the stimulator device 600 is shown to be applied to the target location on the user's neck as described herein. For reference, FIG. 10 shows the locations of the following vertebrae: first cervical vertebra 602, the fifth cervical vertebra 604, the sixth cervical vertebra 606, and the seventh cervical vertebra 608.

Of course, it will be recognized that the vagus nerve may be stimulated through other mechanisms. For example, auricular vagal nerve stimulation involves stimulation of the auricular branch of the vagus nerve, often termed the Alderman's nerve or Arnold's nerve. This nerve may be stimulated through the transcutaneous systems and methods described herein by transmitting electrical impulses through the outer skin surface of the user's ear to the auricular branch of the vagus nerve.

### Systems of the Present Disclosure

Referring now to FIG. 11, a system 700 for stimulating a nerve in a user, such as the vagus nerve, includes a stimulator 712, which may include one or more electrodes 714, a pulse generator 716 and an energy source 712. Stimulator 700 may also include one or more sensors 711, such as the position sensors described above. Electrodes 714, sensors 711, pulse generator 716 and energy source 812 may all be housed in a single housing, as described in detail above. In an alternative embodiment, electrodes 714 and/or sensors 711 are disposed separately from energy source 712 and pulse generator 716. Electrodes 714 and/or sensors 711 may be coupled to these components via wired connections or wirelessly. In the latter configuration, electrodes 714 and/or sensors 711 may include suitable electronic components coupled thereto to receive the electrical impulse(s) from pulse generator 716 and to apply those electrical impulse(s) through electrodes 714 to the user. Such electronic components may include, for example, a wireless receiver or similar component that receives the signal from a wireless transmitter coupled to pulse generator 716.

In still another embodiment, pulse generator 716 and energy source 712 are coupled to each other, either wirelessly, via wired connections, or directly in a housing that contains both components. This housing may, for example, include a wireless transmitter and may be worn by the user in manners known to those skilled in the art, so that the signal can be transmitted from the housing to electrodes 714.

System 700 further includes a controller 718 that is coupled to stimulator 702 and may be used to select or set parameters for the stimulation protocol (amplitude, frequency, pulse width, burst number, electrode positioning etc.), the treatment regimen discussed above (i.e., duration and number of doses, etc.) or alert the user as to the need to use or adjust the stimulator (i.e., an alarm). Controller 718 may be directly coupled to stimulator 702 via wired connectors or within the same housing, or it may be wirelessly coupled to stimulator 702.

Significant portions of the control of the vagus nerve stimulation may reside in controller components that are physically separate from stimulator 702. In this embodiment, separate components of the controller 718 and stimulator 702 generally communicate with one another wirelessly. Thus, the use of wireless technology avoids the inconvenience and distance limitations of interconnecting cables.

In certain embodiments, system 700 may further include one or more mobile device(s) 720 that either couple controller 718 to stimulator 702 or vice versa. Mobile device 720 may comprise a mobile phone, such as a smartphone, wearable electronic device, such as a smartwatch, iPad, laptop computer or any other mobile device having a computing function and wireless transmission technology.

In certain embodiments, system includes a suitable user interface 810 and a computer-readable storage device and/or one or more software applications that allow a user to input current user status information into controller 718. User interface 810 may be located on, for example, stimulator 702, one or more mobile devices 720 or controller 718. The mobile device(s) 720 or the stimulator 702 may include an alert or other alarm that reminds the user to input user status information on a regular time schedule. The user status information may include, for example, a current level of pain, a satisfaction level, a current mood, an amount of recent medication use (e.g., pain medication), a perceived activity level, the amount of sleep that the user has recently received or any other data related to the user's general health or recovery. This user status information is stored within controller 718 and may be displayed in a variety of different forms for the user: list form, graphical form, activity reports and the like. The user status information allows the user (and the prescribing physician) to document the user status information, and it may provide historical trends of this information (e.g., have pain levels or medication use gone down over time) to provide a more holistic picture of his/her progress with the therapy regimen.

In certain embodiments, controller 718 includes a processor that correlates the user status information with other data received from sensors 722, with the parameters of the electrical impulse and/or the overall treatment protocol (i.e., the intensity of the electrical impulse, the duration of single doses, or the number of single doses in total, or over a period of time, such as doses/day, doses/week or the like).

The physiological parameters that may include, but are not limited to, heart rate and variability, ECG, respiration depth and rate, core temperature, hydration, blood pressure, brain function, oxygenation, skin impedance, and skin temperature. Alternatively, the user status information may be correlated with certain parameters of the treatment regimen, such as the duration, amplitude and/or frequency of each single dose applied by the stimulator, the number of single doses applied by the stimulator over certain intervals of time (e.g., doses per day, per week, per month, etc.), the specific locations in which stimulation was applied (i.e., the relative position of the electrodes and the target nerve) and the like.

The processor may be configured to allow the display of this correlated information on the mobile device so that the user and/or physician can compare and track the user status information with the physiological parameters and/or the actual treatment parameters. This provides valuable data to both the user and the physician to help them visualize the effectiveness of the stimulation therapy or to allow them to modify the stimulation therapy regimen to optimize its benefits to the users. For example, the processor may determine that the therapy was more effective when the user applied the single doses for a longer duration or at a higher amplitude. In another example, the processor may determine that the therapy was more effective when the user included certain intervals between doses (e.g., 4-6 hours), or applied a certain number of doses per day or week. In yet another example, the processor may determine that the therapy was more effective when the relative positions of the electrodes and the target nerve were within a certain range. In this latter embodiment, the data could be used to demonstrate that the user received more effective therapy if the electrodes were positioned close enough to the target nerve to achieve effective stimulation thereof. In addition, the processor could pinpoint the distance between the electrodes and the target nerve wherein the stimulation becomes less effective.

In addition, this provides a historical record of this effectiveness so that the user does not have to remember the user status information at, for example, follow-up visits with the physician. For example, if the user sees that certain dosing levels and/or electrode positions of the device (and/or dosing levels that substantially track the prescribing physician's recommendations) correlate with lower pain levels, higher satisfaction, better moods, etc., the user will understand that compliance with the therapy regimen (e.g., routine, timing and duration) provides better outcomes. This understanding may provide better user compliance with the therapy regimen.

One or more of the mobile device(s) 702 preferably includes one or more software applications that display information that enhances the user experience with stimulator 702 and enables the user to track the progress he/she has made with the therapy regimen. For example, upon opening the application and creating a profile, the user may be prompted to provide baseline information on user status, such as mood, pain-level, prescribed medications and the like. The software application may also be configured to prompt the user to set goals or milestones for his/her treatment, such as pain-free activities. The software application may provide a dashboard or similar display that provides a summary of the data that has been collected during the therapy regimen. This summary data may include, for example, progress towards milestones or goals achieved, progress on recovery, such as pain levels, emotional state and/or activity levels and the like. This information may help the user avoid recovery setbacks and improve compliance with the therapy regimen.

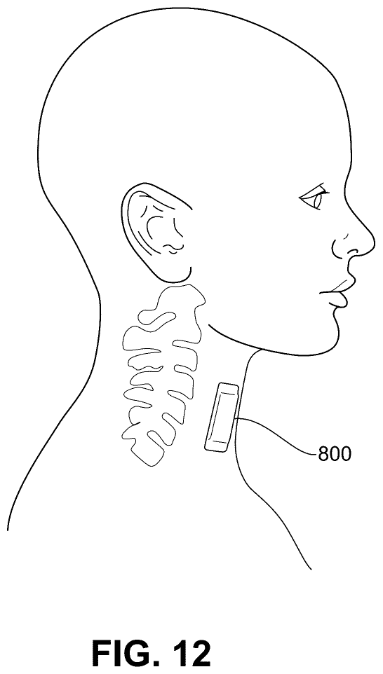

FIGS. 12A-12K illustrates one embodiment of a user interface 850 for use with system 700. Referring now to FIG. 12A, when the user opens up the software application, there is a screen or page 850 that provides instructions for wirelessly pairing the stimulator with the application. These instructions may include a prompt to input an authorization code that allows the stimulator to pair with the application. This authorization code may be the same, or different, from the authorization code that the user submits in order to fill or refill the stimulator with a certain number of authorized or prescribed doses for treatment.

Referring now to FIG. 12B, a profile screen 860 may include a variety of personal information inputted by the user, such as name, gender, address, email address, age, or the symptoms, disorder or disease being treated. The profile screen 860 may provide a prompt to set up a user account. In certain embodiments, a symptom screen 862 may provide a variety of different symptoms for the user to select from. For example, in certain non-medical or wellness applications, the user may select stress/anxiety, lack of sleep, mental or physical performance, mood or others (see FIG. 12J).

Referring again to FIG. 12B, profile screen 860 may also include a user status page (not shown) that includes various prompts for the user to enter certain status information at the beginning, during or between stimulation sessions. As discussed above, the status information may include general status information, such as mood, level of pain, or the like, or more detailed status information specifically related to the user's disorder or disease. In one such example, the stimulator may be used for treatment of addiction, such as opioid use disorder or others. In this example, the user status information may include, any drugs taken prior to, or during, the time period of the treatment, desires or cravings to take drugs, withdrawal symptoms, tolerance and the like. In another example, the stimulator may be used to treat headache, such as migraine, cluster headache, tension headache, PTSD or the like. In this example, the user status information may include pain level or the location of the pain (i.e., one side of head, diffuse, etc.), light, noise or smell sensitivities, nausea, vomiting, loss of appetite, fatigue, dizziness or blurred vision and the like. This status information is stored within the application and may be correlated with the treatment parameters.

Referring now to FIG. 12C, the software application may include an account page 870 that allows the user to view his account (i.e., payments made for prescriptions and the like), view pages that provide information on how to operate the device, contact customer support through the software application or other methods (phone numbers, emails, etc.), or review FAQs that are provided in the software application. The account page 870 may also include a refill page 872 that allows the user to refill the stimulator, i.e., increase the number of single doses and/or duration of time that the stimulator is authorized to apply to the user. This refill page 872 may be linked, for example, to a caregiver's processing device such that the caregiver can provide authorization for such refill. The refill page 872 may also be linked to a payment screen that allows the user to pay for the refill.

Account page 870 may also include a device settings page 874 that provides information on the current device settings (intensity, duration, waveform, frequency, etc.). Alternatively, or in addition, the device setting pages 874 may allow the user to adjust the device settings from the mobile application (rather than directly on the device). This provides a more convenient method for the user to adjust settings. For example, the user may adjust the settings prior to placing the device against his/her skin surface so that the user only needs to hold the device against the skin to apply the stimulation therapy (rather than also adjusting settings at the same time). Alternatively, the user may find it easier to adjust the settings during stimulation from the application than on the device itself.

Account page 870 may also include a data sharing screen 876 that allows the user to share certain data in the application with, for example, a caregiver. In some embodiments, this data will automatically be shared with the caregiver. In other embodiments, the user may select the type of data that is shared.

Referring now to FIG. 12I, the software application may include a stimulation screen 880 that provides information during a stimulation session and/or allows the user to adjust a parameter of stimulation during the session. For example, stimulation screen 880 may include a time counter 882 that tracks the duration of the session and provides a time countdown for the user to visualize how much time is left in the stimulation session. Stimulation screen 880 may also include an intensity display 884 that indicates the current intensity level of the stimulation session and/or allows the user to adjust the intensity during the stimulation session.

Referring now to FIG. 12D, a home screen 820 may include a variety of data and information for the user. In this embodiment, home screen 820 includes an overview page 822, an analysis page 824 and a history page 826. Overview page 822 includes a device connection indicator 832, a schedule 834 for when the user should apply the next dose of electrical stimulation, and a schedule 836 of how many doses and/or how much time are left on the user's prescription. The overview page 822 may also include information such as percentage of battery life, and whether the device is connected with the software application. The application may also include alerts that can be turned ON to remind the user that it is time for a dose according to the treatment regimen prescribed by the physician and/or agreed to by the user. The physician may also have the ability edit this reminder feature in the event that the treatment regimen is changed.

The stimulator 702 may be configured to deactivate and automatically turn OFF and not deliver any further doses when either the number of doses or number of days left reaches zero. In one embodiment, the stimulator 702 requires a new authorization code in order to turn back ON once it has been deactivated. A more complete description of this authorization code can be found in co-pending, commonly assigned U.S. patent application Ser. No. 16/229,299, filed Dec. 21, 2018 and U.S. Pat. No. 17,002,347, filed Aug. 25, 2022, the complete disclosures of which are incorporated herein by reference in their entirety for all purposes.

Referring now to FIGS. 12E and 12K, analysis page 824 may provide a variety of data and information that allows the user to track usage of the device and/or compare the usage of the device over time. For example, analysis page 824 provides information related to the change in the number of stimulations applied by the device over a time period, such as a week, month, six months, or a year (838). These stimulations may be broken up into single doses, multiple doses/day, or the like. Analysis page 824 also provides information related to the average, maximum or minimum intensity or amplitude of the electrical signal applied over a time period, such as a week, month, six months, or a year (840).

In certain embodiments, analysis page 824 may provide additional information, such as a comparison between user status information inputted by the user and/or physiological parameters measured by one of the sensors with the actual treatment parameters (see FIG. 12G). This may, for example, indicate that a decrease in number of stimulations over a weekly period resulted in a reduced in treatment effectiveness, which may be evidenced by changes in user status information (e.g., more pain, increased pain medication, change in mood or the like) and/or changes in physiological parameters, such as those parameters associated with the symptoms of the disorder or disease being treated.

FIG. 12F illustrates one example wherein the user has inputted status information 842 related to their mood at certain time points. This data can be broken up into certain categories, e.g., joyful, happy, relaxed, indifferent, etc. These categories may be automatically included in the software application, or the application may allow the user to input their own categories. With each category, the user may be able to rank his/her mood with respect to that category on a scale, or 1-5, 1-10, 1-20, 1-100 or the like. The application may be further configured to average the user's rankings of moods over a period of time. This information can then be compared to the actual parameters of stimulation and/or the treatment paradigm that has been applied by the user over that time period.

For example, analysis page 824 may indicate that the intensity level of the stimulation has decreased over a time period. This reduction in intensity may be, for example, correlated with a change in mood or another user status data point (e.g., less joyful, more pain, etc.). In this instance, the software application may be configured to automatically adjust the intensity level of the stimulation and/or simply recommend to the user that he/she increase the intensity level by a certain amount.

In another example, analysis page 824 may indicate that the number of single doses of stimulation or the duration of each single dose has decreased over a time period. This reduction in duration or number of doses may be, for example, correlated with a change in mood or another user status data point (e.g., less joyful, more pain, etc.). In this instance, the software application may be configured to automatically adjust the intensity level of the stimulation and/or simply recommend to the user that he/she increase the intensity level by a certain amount.

Referring now to FIG. 12H, the software application may include a treatment regimen page 890 that provides separate pages depending on the type of treatment, e.g., acute, chronic or preventative. These pages may contain a variety of useful information related to the history of the device parameters applied for each treatment regimen, e.g., average intensity, average duration, cumulative number of single doses applied, number of single doses applied over a particular time period and the like. These pages may also provide screens or prompts that allow the user to input status information, such as pain on a scale before and after stimulation. As discussed above, this status information may be correlated with the actual device parameters used to provide the user or caregiver with valuable information as to the treatment effectiveness.

Referring now to FIG. 12J, the software application may include a symptom selection page 895 that allows the user to select one or more symptoms to treat with the stimulator. In certain applications, the symptom selection page 895 may include general wellness symptoms, such as reducing stress and anxiety, enhancing relaxation, improving sleep, promoting mental health, recovery and wellness and generally improving the health and wellbeing of a user. Alternatively, the user may select certain functions that improve energy, concentration and mood, increasing mental or physical performance and the like. For example, the devices can be configured to increase cognitive performance, skill proficiency, judgement, vigilance, attention and memory by inducing neuroplasticity, improving neurobehavioral outcomes, enhancing focus and mitigating fatigue. Examples include enhancing learning and skill acquisition, such as second language learning vocabulary acquisition and/or enhancing cognitive performance in extreme environments, such as extreme stressors, multiday transoceanic operational and logistic flights, long duration remotely piloted aircraft missions and the like.

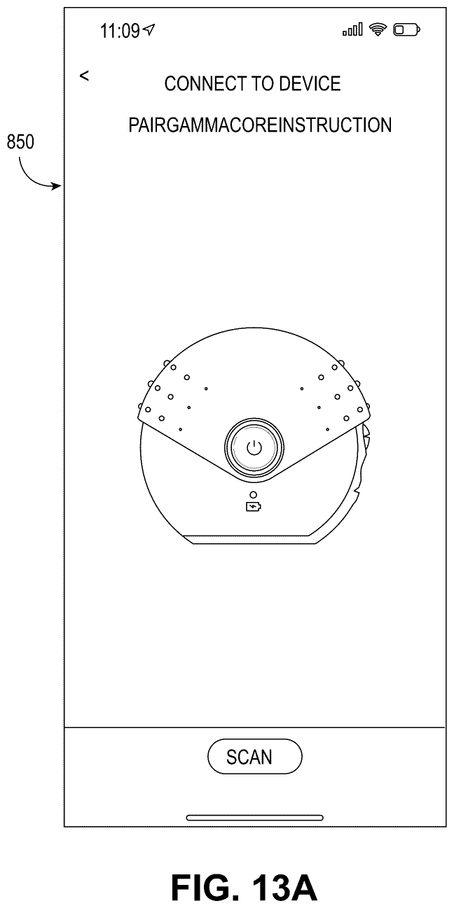

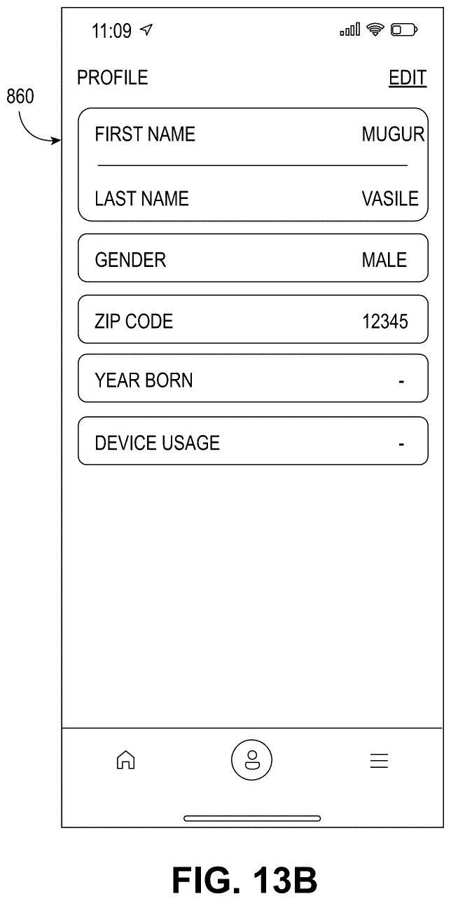

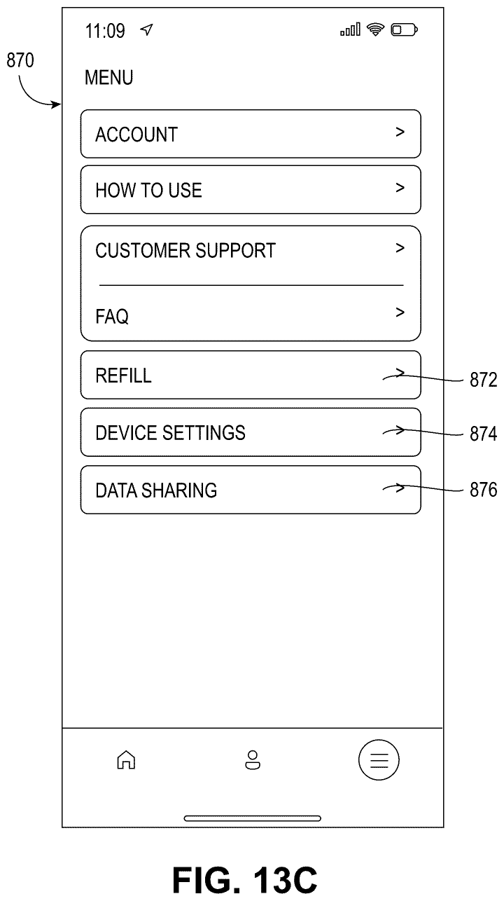

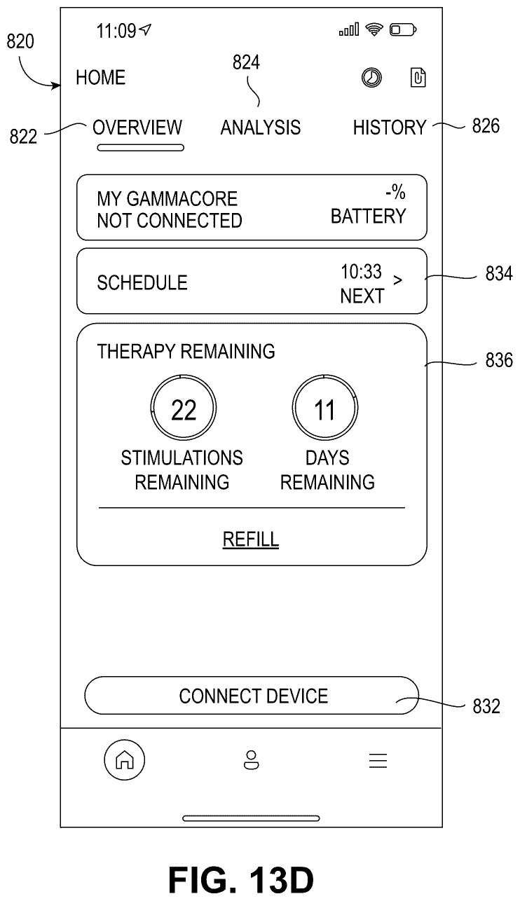

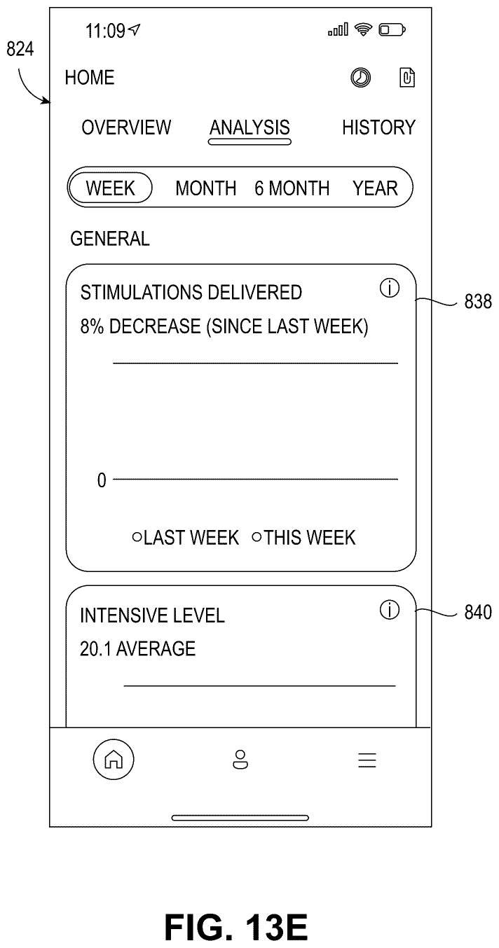

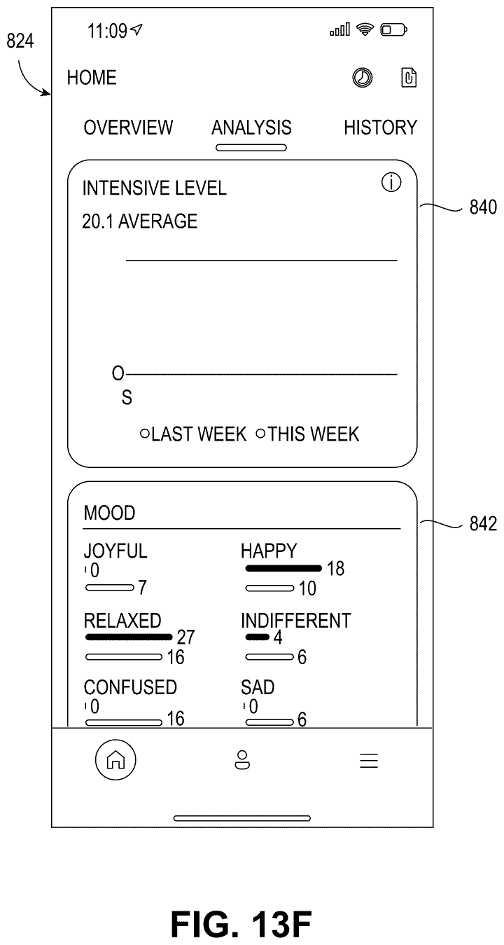

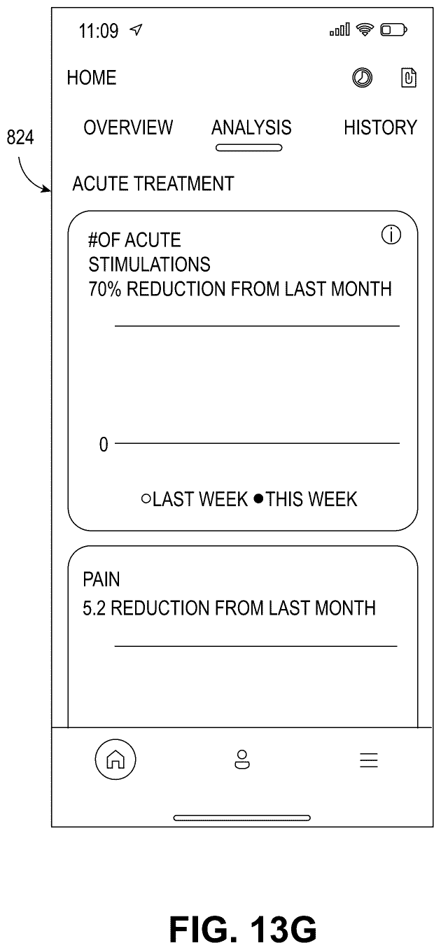

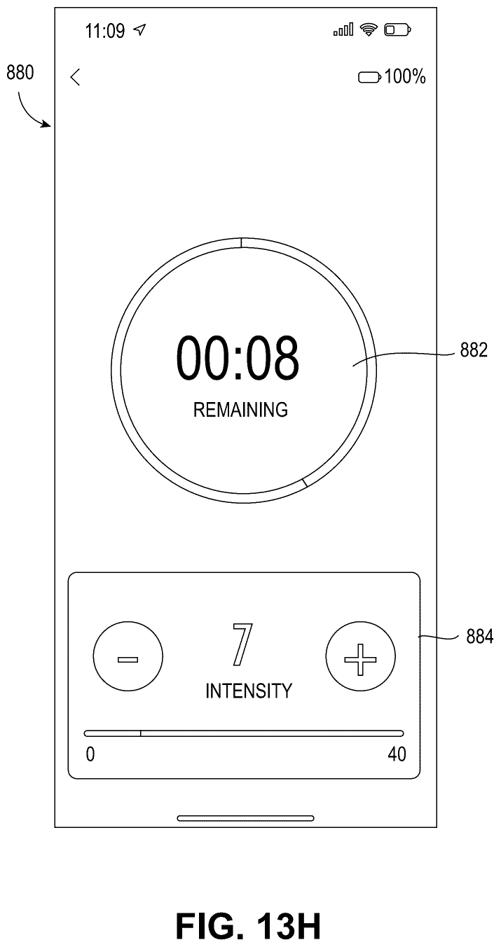

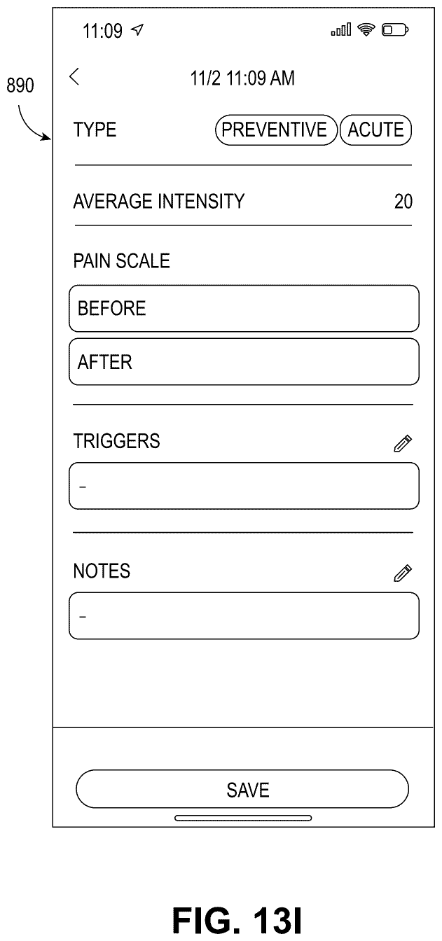

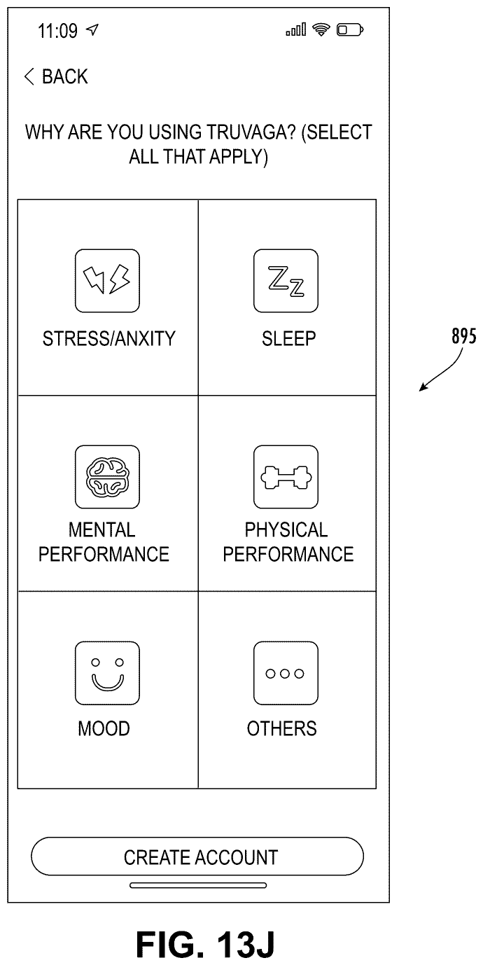

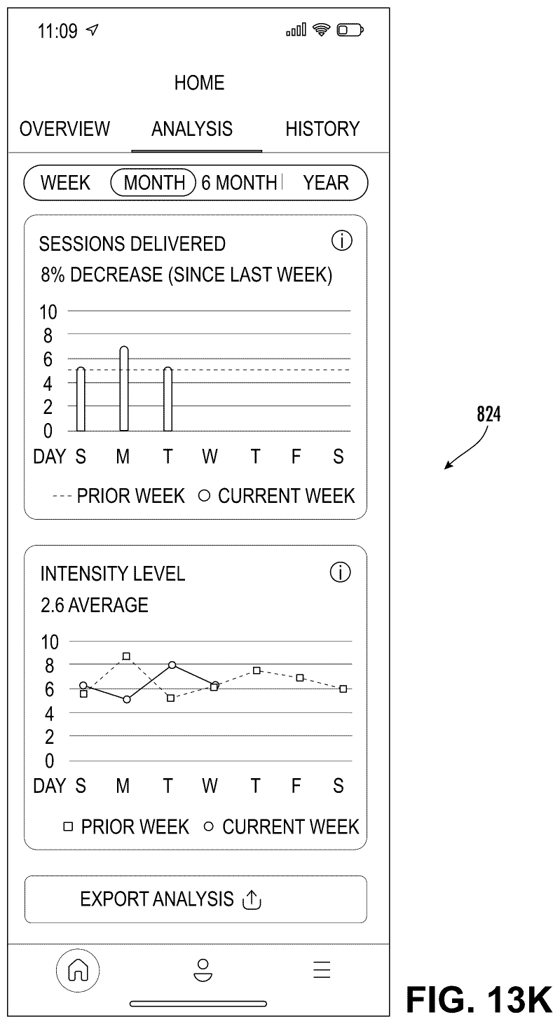

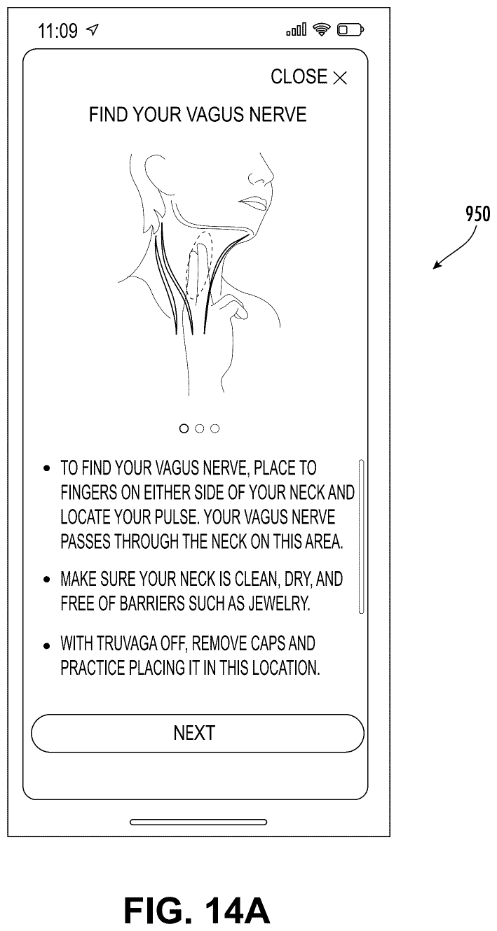

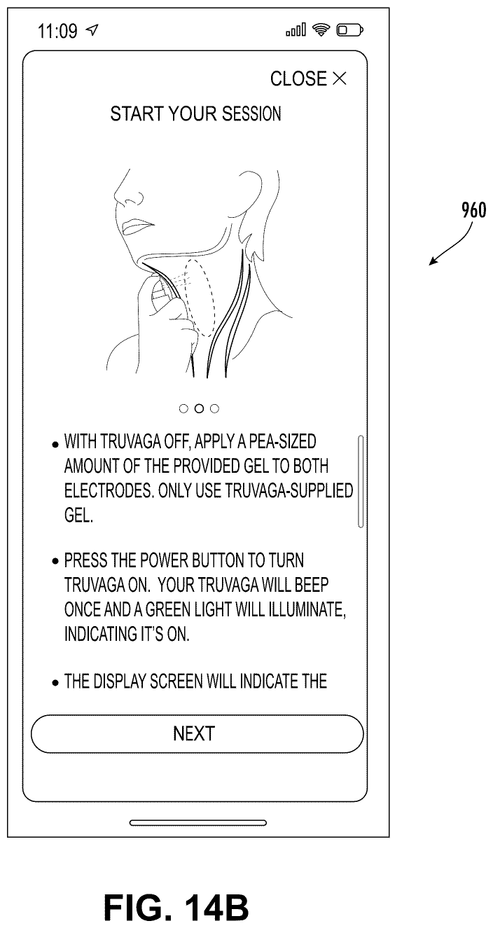

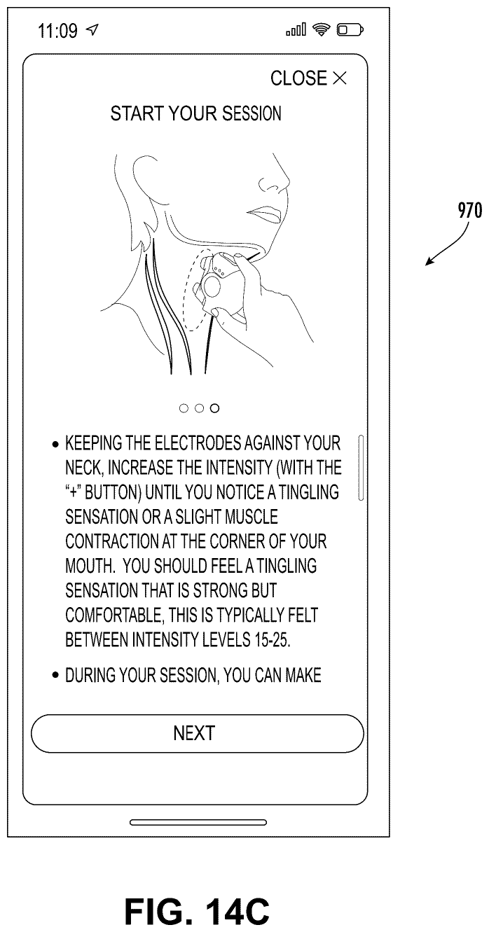

Referring now to FIGS. 13A-13C, the software application may include one or more pages that provide detailed directions on how to position the stimulator against the selected nerve to optimize performance. For example, in FIG. 13A, a first page 950 is illustrated that provides information on how to find the vagus nerve. In FIG. 15B, a second page 960 is illustrated that provides information on how to start the stimulation session by, for example, applying conductive gel to the target site and turning ON the stimulation device. In FIG. 15C, a third page 970 is illustrated that provides information on how to actually conduct the treatment session by, for example, maintaining contact between the electrodes and the skin surface and increasing intensity until the user feels a tingling sensation or slight muscle contraction.

Stimulator 702 may also transmit other information to mobile device(s) 720, controller 718 or directly to a separate processing device (e.g., one operated by a caregiver). This information may include, for example, error data and/or incomplete circuit data produced by stimulator 702. For example, if the stimulator 702 produces an incomplete circuit data, this could mean that the user requires assistance in placement of the electrodes. If the stimulator 702 produces error data, this could mean that the user requires assistance troubleshooting stimulator 702.

In certain applications, system 700 may include a user or user software application and a separate caregiver (e.g., physician) software application. In an exemplary embodiment, the physician software application may be configured to allow the data from individual users to be aggregated together to form data across a plurality of different users. This aggregated data may allow the physician to determine the overall effectiveness of the therapy across multiple users. In addition, it may allow the physician to better understand the impact of usage of the device with the effectiveness of the therapy. For example, the data may show that increased usage of the device and/or improved compliance with the therapy regimen increases overall effectiveness or reduction in pain.

In certain embodiments, the physician software application may be configured to automatically produce reports of complied data from system 700 and/or stimulator 702 that may include, for example, user compliance with the therapy regimen, user status data (e.g., pain), physiological parameters and/or the actual treatment parameters. The software application may be designed to aggregate these data into single reports that allow the physician to easily compare, for example, treatment parameters with pain, user satisfaction, medication user, activity levels and the like.

System 700 may further include a recharging outlet or station (also not shown) configured to receive a rechargeable battery. Alternatively, the battery may comprise an outlet or other coupling element for directly charging the battery with a suitable electrical connector (i.e., without removing the battery from the stimulator housing). Providing a rechargeable battery that may be easily switched out allows 24 hour use of the device, which may increase the effectiveness of the device. In other embodiments, the energy source may be located exterior to the housing and either directly connected thereto with wires or other electrical connections, or wireless coupled to the housing via a suitable wireless energy transmitter/receiver device.

In certain embodiments, the energy source includes a data storage component (not shown) coupled to a processor within stimulation device 710. The processor is configured to transfer data, such as motion data, usage levels, or any other data collected by the processor, to the data storage component. The data storage component may be accessed by a separate processor external to the stimulation device (e.g., in the mobile device or a separate processing device) when the battery is removed for recharging. This allows large amounts of data to be transferred from the stimulation device to the mobile device, i.e., larger amounts of data that may be possible through wireless transmission alone.

In addition to position sensors 711, system 700 may further include one or more additional sensors (not shown) used for detecting certain physiological parameters of the user based on the stimulation of the nerve. The preferred sensors will include ones ordinarily used for ambulatory monitoring. For example, the sensors may comprise those used in conventional Holter and bedside monitoring applications, for monitoring heart rate and variability, ECG, respiration depth and rate, core temperature, hydration, blood pressure, brain function, oxygenation, skin impedance, and skin temperature. The sensors may be embedded in garments or placed in sports wristwatches, as currently used in programs that monitor the physiological status of soldiers [G. A. SHAW, A. M. Siegel, G. Zogbi, and T. P. Opar. Warfighter physiological and environmental monitoring: a study for the U.S. Army Research Institute in Environmental Medicine and the Soldier Systems Center. MIT Lincoln Laboratory, Lexington MA. 1 Nov. 2004, pp. 1-141]. The ECG sensors should be adapted to the automatic extraction and analysis of particular features of the ECG, for example, indices of P-wave morphology, as well as heart rate variability indices of parasympathetic and sympathetic tone. Measurement of respiration using noninvasive inductive plethysmography, mercury in silastic strain gauges or impedance pneumography is particularly advised, in order to account for the effects of respiration on the heart. A noninvasive accelerometer may also be included among the ambulatory sensors, in order to identify motion artifacts. An event marker may also be included in order for the user to mark relevant circumstances and sensations.

For brain monitoring, the sensors may comprise ambulatory EEG sensors [CASSON A, Yates D, Smith S, Duncan J, Rodriguez-Villegas E. Wearable electroencephalography. What is it, why is it needed, and what does it entail? IEEE Eng Med Biol Mag. 29 (3,2010): 44-56] or optical topography systems for mapping prefrontal cortex activation [Atsumori H, Kiguchi M, Obata A, Sato H, Katura T, Funane T, Maki A. Development of wearable optical topography system for mapping the prefrontal cortex activation. Rev Sci Instrum. 2009 April; 80 (4): 043704]. Signal processing methods, comprising not only the application of conventional linear filters to the raw EEG data, but also the nearly real-time extraction of non-linear signal features from the data, may be considered to be a part of the EEG monitoring [D. Puthankattil SUBHA, Paul K. Joseph, Rajendra Acharya U, and Choo Min Lim. EEG signal analysis: A survey. J Med Syst 34 (2010): 195-212]. In the present application, the features would include EEG bands (e.g., delta, theta, alpha, beta).

For any given position of the stimulator relative to the vagus nerve, it is also possible to infer the amplitude of the electric field that it produces in the vicinity of the vagus nerve. This is done by calculation or by measuring the electric field that is produced by the stimulator as a function of depth and position within a phantom that simulates the relevant bodily tissue [Francis Marion MOORE. Electrical Stimulation for pain suppression: mathematical and physical models. Thesis, School of Engineering, Cornell University, 2007; Bartosz SAWICKI, Robert Szmurło, Przemysław Płonecki, Jacek Starzyński, Stanisław Wincenciak, Andrzej Rysz. Mathematical Modelling of Vagus Nerve Stimulation. pp. 92-97 in: Krawczyk, A. Electromagnetic Field, Health and Environment: Proceedings of EHE'07. Amsterdam, IOS Press, 2008]. Thus, in order to compensate for movement, the controller may increase or decrease the amplitude of the output from the stimulator (u) in proportion to the inferred deviation of the amplitude of the electric field in the vicinity of the vagus nerve, relative to its desired value.

Various corresponding structures, materials, acts, and equivalents of all means or step plus function elements in various claims below are intended to include any structure, material, or act for performing the function in combination with other claimed elements as specifically claimed. Various embodiments were chosen and described in order to best explain various principles of this disclosure and various practical applications thereof, and to enable others of ordinary skill in a pertinent art to understand this disclosure for various embodiments with various modifications as are suited to a particular use contemplated.

Various diagrams depicted herein are illustrative. There can be many variations to such diagrams or steps (or operations) described therein without departing from various spirits of this disclosure. For instance, various steps can be performed in a differing order or steps can be added, deleted or modified. All of these variations are considered a part of this disclosure. People skilled in an art to which this disclosure relates, both now and in future, can make various improvements and enhancements which fall within various scopes of various claims which follow.

For example, it will also be appreciated that some the devices and methods can be applied to other tissues and nerves of the body, including but not limited to other parasympathetic nerves, sympathetic nerves, spinal or cranial nerves, muscles, peripheral nerve stimulation, spinal cord stimulation, neuromuscular electrical stimulation (NMES) or other nerves, such as abdominal aortic plexus, abducens nerve, accessory nerve, accessory obturator nerve, alderman's nerve, anococcygeal nerve, ansa cervicalis, anterior interosseous nerve, anterior superior alveolar nerve, auerbach's plexus, auriculotemporal nerve, axillary nerve, brachial plexus, buccal branch of the facial nerve, buccal nerve, cardiac plexus, cavernous nerves, cavernous plexus, celiac ganglia, cervical branch of the facial nerve, cervical plexus, chorda tympani, ciliary ganglion, coccygeal nerve, cochlear nerve, common fibular nerve, common palmar digital nerves of median nerve, deep branch of the radial nerve, deep fibular nerve, deep petrosal nerve, deep temporal nerves, diagonal band of broca, digastric branch of facial nerve, dorsal branch of ulnar nerve, dorsal nerve of clitoris, dorsal nerve of the penis, dorsal scapular nerve, esophageal plexus, ethmoidal nerves, external laryngeal nerve, external nasal nerve, facial nerve, femoral nerve, frontal nerve, gastric plexuses, geniculate ganglion, genital branch of genitofemoral nerve, genitofemoral nerve, glossopharyngeal nerve, greater auricular nerve, greater occipital nerve, greater petrosal nerve, hepatic plexus, hypoglossal nerve, iliohypogastric nerve, ilioinguinal nerve, inferior alveolar nerve, inferior anal nerves, inferior cardiac nerve, inferior cervical ganglion, inferior gluteal nerve, inferior hypogastric plexus, inferior mesenteric plexus, inferior palpebral nerve, infraorbital nerve, infraorbital plexus, infratrochlear nerve, intercostal nerves, intercostobrachial nerve, intermediate cutaneous nerve, internal carotid plexus, internal laryngeal nerve, interneuron, jugular ganglion, lacrimal nerve, lateral cord, lateral cutaneous nerve of forearm, lateral cutaneous nerve of thigh, lateral pectoral nerve, lateral plantar nerve, lateral pterygoid nerve, lesser occipital nerve, lingual nerve, long ciliary nerves, long root of the ciliary ganglion, long thoracic nerve, lower subscapular nerve, lumbar nerves, lumbar plexus, lumbar splanchnic nerves, lumboinguinal nerve, lumbosacral plexus, lumbosacral trunk, mandibular nerve, marginal mandibular branch of facial nerve, masseteric nerve, maxillary nerve, medial cord, medial cutaneous nerve of arm, medial cutaneous nerve of forearm, medial cutaneous nerve, medial pectoral nerve, medial plantar nerve, medial pterygoid nerve, median nerve, meissner's plexus, mental nerve, middle cardiac nerve, middle cervical ganglion, middle meningeal nerve, motor nerve, muscular branches of the radial nerve, musculocutaneous nerve, mylohyoid nerve, nasociliary nerve, nasopalatine nerve, nerve of pterygoid canal, nerve to obturator internus, nerve to quadratus femoris, nerve to the piriformis, nerve to the stapedius, nerve to the subclavius, nervus intermedius, nervus spinosus, nodose ganglion, obturator nerve, oculomotor nerve, olfactory nerve, ophthalmic nerve, optic nerve, otic ganglion, ovarian plexus, palatine nerves, palmar branch of the median nerve, palmar branch of ulnar nerve, pancreatic plexus, patellar plexus, pelvic splanchnic nerves, perforating cutaneous nerve, perineal branches of posterior femoral cutaneous nerve, perineal nerve, petrous ganglion, pharyngeal branch of vagus nerve, pharyngeal branches of glossopharyngeal nerve, pharyngeal nerve, pharyngeal plexus, phrenic nerve, phrenic plexus, posterior auricular nerve, posterior branch of spinal nerve, posterior cord, posterior cutaneous nerve of arm, posterior cutaneous nerve of forearm, posterior cutaneous nerve of thigh, posterior scrotal nerves, posterior superior alveolar nerve, proper palmar digital nerves of median nerve, prostatic plexus (nervous), pterygopalatine ganglion, pudendal nerve, pudendal plexus, pulmonary branches of vagus nerve, radial nerve, recurrent laryngeal nerve, renal plexus, sacral plexus, sacral splanchnic nerves, saphenous nerve, sciatic nerve, semilunar ganglion, sensory nerve, short ciliary nerves, sphenopalatine nerves, splenic plexus, stylohyoid branch of facial nerve, subcostal nerve, submandibular ganglion, suboccipital nerve, superficial branch of the radial nerve, superficial fibular nerve, superior cardiac nerve, superior cervical ganglion, superior ganglion of glossopharyngeal nerve, superior ganglion of vagus nerve, superior gluteal nerve, superior hypogastric plexus, superior labial nerve, superior laryngeal nerve, superior lateral cutaneous nerve of arm, superior mesenteric plexus, superior rectal plexus, supraclavicular nerves, supraorbital nerve, suprarenal plexus, suprascapular nerve, supratrochlear nerve, sural nerve, sympathetic trunk, temporal branches of the facial nerve, third occipital nerve, thoracic aortic plexus, thoracic splanchnic nerves, thoraco-abdominal nerves, thoracodorsal nerve, tibial nerve, transverse cervical nerve, trigeminal nerve, trochlear nerve, tympanic nerve, ulnar nerve, upper subscapular nerve, uterovaginal plexus, vagus nerve, ventral ramus, vesical nervous plexus, vestibular nerve, vestibulocochlear nerve, zygomatic branches of facial nerve, zygomatic nerve, zygomaticofacial nerve, or zygomaticotemporal nerve.

1. A system for stimulating a nerve within a user, the system comprising:
a stimulator comprising an electrode configured for contacting the outer skin surface at, or near the target location;
an energy source coupled to the stimulator, wherein the energy source is configured to generate at least one electrical impulse and to transmit the at least one electrical impulse transcutaneously from the electrode through the outer skin surface of the user to a selected nerve in the user adjacent to, or near, the target location; and
wherein the electrical impulse is sufficient to improve a general wellness of the user.

2. The system of claim 1, wherein the electrical impulse is sufficient to reduce a stress of the user.

3. The system of claim 1, wherein the electrical impulse is sufficient to reduce anxiety of the user.

4. The system of claim 1, wherein the electrical impulse is sufficient to increase an energy level of the user.

5. The system of claim 1, wherein the stimulator comprises a housing, wherein the electrode is coupled to the housing.

6. The system of claim 5, wherein the energy source is disposed within the housing.

7. The system of claim 6, further comprising a signal generator disposed within the housing and electrically coupled to the energy source and the electrode.

8. The system of claim 5, wherein the housing comprises a power button disposed on a surface of the housing, wherein the power button comprises a user interface.

9. The system of claim 8, wherein the power button comprises a substantially circular main body with first and second arms extending therefrom, wherein the first and second arms comprise openings therethrough.

10. The system of claim 8, further comprising a support member between the power button and the housing, wherein the support member comprises a main body having at least one support arm extending therefrom.

11. The system of claim 10, wherein the support member comprises a first surface coupled to the power button, the first surface tapering inwardly toward the housing.

12. The system of claim 11, wherein the support member further comprises a second surface coupled to the housing, the second surface having a recessed portion.

13. The system of claim 1, wherein the electrical impulse comprises pulses having a frequency of about 1 kHz to about 20 KHz.

14. The system of claim 1, wherein the electrical impulse comprises bursts of pulses, with each burst having a frequency of about 1 to about 100 bursts per second and each pulse has a duration of about 50 to about 1000 microseconds in duration.

15. The system of claim 14, wherein the bursts each comprise about 2 to 20 pulses and the bursts are separated by an inter-burst period that comprises zero pulses.

16. The system of claim 1, wherein the nerve is a vagus nerve.

17. The system of claim 1, wherein the electrode is configured for contacting an outer skin surface of the neck of the user.

18. A system for stimulating a nerve within a user, the system comprising:
a stimulator comprising an electrode configured for contacting the outer skin surface at, or near the target location;
an energy source coupled to the stimulator, wherein the energy source is configured to generate at least one electrical impulse and to transmit the at least one electrical impulse transcutaneously from the electrode through the outer skin surface of the user to a selected nerve in the user adjacent to, or near, the target location; and
wherein the electrical impulse is sufficient to improve a cognitive performance of the user.

19. The system of claim 18, wherein the electrical impulse is sufficient to increase a memory of the user.

20. The system of claim 18, wherein the electrical impulses is sufficient to reduce a fatigue of the user.

21. The system of claim 18, wherein the electrical impulse is sufficient to increase a language acquisition skill of the user.

22. The system of claim 18, wherein the electrical impulse is sufficient to increase an attention span of the user.

23. The system of claim 18, wherein the electrical impulse is sufficient to increase a focus of the user.

24. The system of claim 18, wherein the stimulator comprises a housing, wherein the electrode is coupled to the housing.

25. The system of claim 24, wherein the energy source is disposed within the housing.

26. The system of claim 24, further comprising a signal generator disposed within the housing and electrically coupled to the energy source and the electrode.

27. The system of claim 24, wherein the housing comprises a power button disposed on a surface of the housing, wherein the power button comprises a user interface.

28. The system of claim 27, wherein the power button comprises a substantially circular main body with first and second arms extending therefrom, wherein the first and second arms comprise openings therethrough.

29. The system of claim 27, further comprising a support member between the power button and the housing, wherein the support member comprises a main body having at least one support arm extending therefrom.

30. The system of claim 29, wherein the support member comprises a first surface coupled to the power button, the first surface tapering inwardly toward the housing.

31. The system of claim 30, wherein the support member further comprises a second surface coupled to the housing, the second surface having a recessed portion.

32. The system of claim 18, wherein the electrical impulse comprises pulses having a frequency of about 1 kHz to about 20 KHz.

33. The system of claim 18, wherein the electrical impulse comprises bursts of pulses, with each burst having a frequency of about 1 to about 100 bursts per second and each pulse has a duration of about 50 to about 1000 microseconds in duration.

34. The system of claim 33, wherein the bursts each comprise about 2 to 20 pulses and the bursts are separated by an inter-burst period that comprises zero pulses.

35. The system of claim 18, wherein the nerve is a vagus nerve.

36. The system of claim 18, wherein the electrode is configured for contacting an outer skin surface of the neck of the user.
